(19) United States
(12) Patent Application Publication
(10) Pub. No.: US 2022/0109117 A1
(43) Pub. Date: Apr. 7, 2022

(54) PHOTOELECTRIC CONVERSION ELEMENT, IMAGING ELEMENT, OPTICAL SENSOR, AND MATERIAL FOR PHOTOELECTRIC CONVERSION ELEMENT

(71) Applicant: FUJIFILM Corporation, Tokyo (JP)

(72) Inventors: Tomoaki YOSHIOKA, Kanagawa (JP); Koichi IWASAKI, Kanagawa (JP)

(73) Assignee: FUJIFILM Corporation, Tokyo (JP)

(21) Appl. No.: 17/551,210

(22) Filed: Dec. 15, 2021

Related U.S. Application Data
(63) Continuation of application No. PCT/JP2020/022336, filed on Jun. 5, 2020.

(30) Foreign Application Priority Data
Jun. 27, 2019 (JP) 2019-119759

(51) Int. Cl. H01L 51/00; C07D 495/14

(52) U.S. Cl. CPC H01L 51/0074; C07D 495/14; H01L 51/4253; H01L 51/0072; H01L 51/0071; H01L 51/0058

(57) ABSTRACT

An object of the present invention is to provide a photoelectric conversion element that exhibits stable performance even though a compositional ratio of a photoelectric conversion film fluctuates in a case of manufacturing the photoelectric conversion film by vapor deposition. In addition, an imaging element, an optical sensor, and a material for a photoelectric conversion element are provided. The photoelectric conversion element of the present invention includes a conductive film, a photoelectric conversion film, and a transparent conductive film in this order, in which the photoelectric conversion film contains a compound represented by Formula (1) and an n-type semiconductor material.

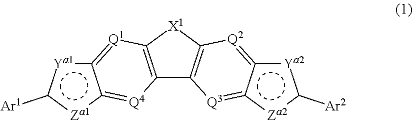

### CROSS-REFERENCE TO RELATED APPLICATIONS

This application is a Continuation of PCT International Application No. PCT/JP2020/022336 filed on Jun. 5, 2020, which claims priority under 35 U.S.C. § 119(a) to Japanese Patent Application No. 2019-119759 filed on Jun. 27, 2019. The above application is hereby expressly incorporated by reference, in its entirety, into the present application.

### BACKGROUND OF THE INVENTION

### 1. Field of the Invention

The present invention relates to a photoelectric conversion element, an imaging element, an optical sensor, and a material for a photoelectric conversion element.

### 2. Description of the Related Art

In recent years, the development of an element (for example, an imaging element) having a photoelectric conversion film has been progressing.

For example, it is disclosed in WO2017/159684A that a photoelectric conversion element has a photoelectric conversion layer containing a predetermined compound.

### SUMMARY OF THE INVENTION

In recent years, along with the demand for improving the performance of imaging elements, optical sensors, and the like, further improvements are required for various characteristics required for photoelectric conversion elements used therein.

For example, due to manufacturing requirements, the photoelectric conversion elements are required to be able to realize stable performance (particularly, dark current characteristics) even though a compositional ratio of a photoelectric conversion film fluctuates in a case of manufacturing the photoelectric conversion film by vapor deposition.

In view of the above circumstances, an object of the present invention is to provide a photoelectric conversion element that exhibits stable performance even though a compositional ratio of a photoelectric conversion film fluctuates in a case of manufacturing the photoelectric conversion film in the photoelectric conversion element by vapor deposition.

Another object of the present invention is to provide an imaging element, an optical sensor, and a material for a photoelectric conversion element.

The present inventors have conducted extensive studies on the above-described problems, and as a result, the inventors have found that it is possible to solve the above-described problems by configurations described below, and have completed the present invention.

[1]

A photoelectric conversion element comprising, in the following order: a conductive film; a photoelectric conversion film; and a transparent conductive film,

in which the photoelectric conversion film contains a compound represented by Formula (1) and an n-type semiconductor material.

[2]

The photoelectric conversion element according to [1], in which the compound represented by Formula (1) is a compound represented by Formula (2).

[3]

The photoelectric conversion element according to [1] or [2], in which the compound represented by Formula (1) is a compound represented by Formula (3).

[4]

The photoelectric conversion element according to [3], in which in Formula (3), X², Y^{b1}, and Y^{b2} represent —S—.

[5]

The photoelectric conversion element according to any one of [1] to [4], in which in Formula, Ar¹ and Ar² each independently represent a polycyclic aromatic hydrocarbon ring group which may have a substituent or a group represented by Formula (R).

[6]

The photoelectric conversion element according to any one of [1] to [5], in which the compound represented by Formula (1) has a molecular weight of 400 to 900.

[7]

The photoelectric conversion element according to any one of [1] to [6], in which the photoelectric conversion film has a bulk hetero structure formed in a state where the compound represented by Formula (1) and the n-type semiconductor material are mixed to each other.

[8]

The photoelectric conversion element according to any one of [1] to [7], further comprising one or more interlayers between the conductive film and the transparent conductive film, in addition to the photoelectric conversion film.

[9]

The photoelectric conversion element according to any one of [1] to [8], in which the n-type semiconductor material includes fullerenes selected from the group consisting of a fullerene and a derivative thereof.

[10]

An imaging element comprising the photoelectric conversion element according to any one of [1] to [9].

[11]

An optical sensor comprising the photoelectric conversion element according to any one of [1] to [9].

[12]

A material for a photoelectric conversion element comprising a compound represented by Formula (1).

According to the present invention, it is possible to provide a photoelectric conversion element that exhibits stable performance even though a compositional ratio of a photoelectric conversion film fluctuates in a case of manufacturing the photoelectric conversion film in the photoelectric conversion element by vapor deposition.

In addition, according to the present invention, it is possible to provide an imaging element, an optical sensor, and a material for a photoelectric conversion element.

### BRIEF DESCRIPTION OF THE DRAWINGS

FIG. 1 is a schematic cross-sectional view showing a configuration example of a photoelectric conversion element.

FIG. 2 is a schematic cross-sectional view showing a configuration example of a photoelectric conversion element.

FIG. 3 is a schematic cross-sectional view of an embodiment of an imaging element.

### DESCRIPTION OF THE PREFERRED EMBODIMENTS

Hereinafter, suitable embodiments of a photoelectric conversion element of the present invention will be described.

In addition, in the present specification, a “substituent” includes a group exemplified by a substituent W described later, unless otherwise specified.

(Substituent W)

A substituent W in the present specification will be described below.

Examples of the substituent W include a halogen atom (such as a fluorine atom, a chlorine atom, a bromine atom, an iodine atom), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heteroaryl group (the heteroaryl group may also be referred to as a heterocyclic group), a cyano group, a hydroxy group, a carboxy group, a nitro group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group (including an anilino group), an ammonio group, an acylamino group, an aminocarbonylamino group, an alkoxycarbonylamino group, an aryloxycarbonylamino group, a sulfamoylamino group, an alkyl or an arylsulfonylamino group, a mercapto group, an alkylthio group, an arylthio group, a heterocyclic thio group, a sulfamoyl group, an alkyl or an arylsulfinyl group, an alkyl or an arylsulfonyl group, an acyl group, an aryloxycarbonyl group, an alkoxycarbonyl group, a carbamoyl group, an aryl or a heterocyclic azo group, an imide group, a phosphino group, a phosphinyl group, a phosphinyloxy group, a phosphinylamino group, a phosphono group, a silyl group, a hydrazino group, a ureido group, and a boronate group (—B(OH)₂). In addition, each of the above-described groups may further have a substituent (for example, one or more groups of each of the above-described groups), as possible. For example, an alkyl group which may have a substituent is also included as a form of the substituent W.

In addition, in a case where the substituent W has a carbon atom, the number of carbon atoms of the substituent W is, for example, 1 to 20.

The number of atoms other than a hydrogen atom included in the substituent W is, for example, 1 to 30.

In addition, in the present specification, unless otherwise specified, the number of carbon atoms of the alkyl group is preferably 1 to 20, more preferably 1 to 10, and even more preferably 1 to 6.

The alkyl group may be any of linear, branched, or cyclic.

Examples of the alkyl group include a methyl group, an ethyl group, an n-propyl group, an i-propyl group, an n-butyl group, a t-butyl group, an n-hexyl group, a cyclopentyl group, and the like.

In addition, the alkyl group may be, for example, a cycloalkyl group, a bicycloalkyl group, or a tricycloalkyl group, and may have a cyclic structure thereof as a partial structure.

In the alkyl group which may have a substituent, a substituent which may be contained in the alkyl group is not particularly limited, an example thereof includes the substituent W, and an aryl group (preferably having 6 to 18 carbon atoms, and more preferably having 6 carbon atoms), a heteroaryl group (preferably having 5 to 18 carbon atoms, and more preferably having 5 and 6 carbon atoms), or a halogen atom (preferably a fluorine atom or a chlorine atom) is preferable.

In addition, in the present specification, unless otherwise specified, the above-described alkyl group is preferable as an alkyl group moiety in the alkoxy group. The alkyl group moiety in the alkylthio group is preferably the above-described alkyl group.

In the alkoxy group which may have a substituent, the substituent which may be contained in the alkoxy group includes the same examples as the substituent in the alkyl group which may have a substituent. In the alkylthio group which may have a substituent, the substituent which may be contained in the alkylthio group includes the same examples as the substituent in the alkyl group which may have a substituent.

In addition, in the present specification, unless otherwise specified, the aryl group is preferably an aryl group having 6 to 18 ring members.

The aryl group may be monocyclic or polycyclic.

The aryl group is preferably, for example, a phenyl group, a naphthyl group, an anthryl group, or a phenanthrenyl group.

In the aryl group which may have a substituent, the substituent which may be contained in the aryl group is not particularly limited, and an example thereof includes the substituent W, an alkyl group which may have a substituent (preferably having 1 to 10 carbon atoms) is preferable, and a methyl group is more preferable.

In addition, in the present specification, unless otherwise specified, the heteroaryl group is preferably a heteroaryl group having a monocyclic or polycyclic ring structure, which contains a heteroatom such as a nitrogen atom, a sulfur atom, an oxygen atom, a selenium atom, a tellurium atom, a phosphorus atom, a silicon atom, and/or a boron atom.

The number of carbon atoms among the ring member atoms of the above-described heteroaryl group is not particularly limited, but is preferably 3 to 18, and more preferably 3 to 5.

The number of heteroatoms among the ring member atoms of the heteroaryl group is not particularly limited, but is preferably 1 to 10, more preferably 1 to 4, and even more preferably 1 to 2.

The number of ring members of the heteroaryl group is not particularly limited, but is preferably 5 to 8, more preferably 5 to 7, and even more preferably 5 and 6.

Examples of the above-described heteroaryl group include a furyl group, a pyridyl group, a quinolyl group, an isoquinolyl group, an acridinyl group, a phenanthridinyl group, a pteridinyl group, a pyrazinyl group, a quinoxalinyl group, a pyrimidinyl group, a quinazolyl group, a pyridazinyl group, a cinnolinyl group, a phthalazinyl group, a triazinyl group, an oxazolyl group, a benzoxazolyl group, a thiazolyl group, a benzothiazolyl group, an imidazolyl group, a benzimidazolyl group, a pyrazolyl group, an indazolyl group, an isoxazolyl group, a benzisoxazolyl group, an isothiazolyl group, a benzisothiazolyl group, an oxadiazolyl group, a thiadiazolyl group, a triazolyl group, a tetrazolyl group, a benzofuryl group, a thienyl group, a benzothienyl group, a dibenzofuryl group, a dibenzothienyl group, a pyrrolyl group, an indolyl group, an imidazopyridinyl group, a carbazolyl group, and the like.

In the heteroaryl group which may have a substituent, the substituent which may be contained in the heteroaryl group is not particularly limited, and an example thereof includes the substituent W.

In addition, in the present specification, the numerical range represented by “to” means a range including numerical values denoted before and after “to” as a lower limit value and an upper limit value.

In the present specification, a hydrogen atom may be a light hydrogen atom (an ordinary hydrogen atom) or a deuterium atom (a double hydrogen atom and the like).

The photoelectric conversion element according to an embodiment of the present invention includes a conductive film, a photoelectric conversion film, and a transparent conductive film in this order, in which the photoelectric conversion film contains a compound represented by Formula (1) (hereinafter, referred to as a “specific compound”) and an n-type semiconductor material.

The mechanism capable of solving the above problems by adopting such a configuration of the photoelectric conversion element according to the embodiment of the present invention is not always clear, but the present inventors speculate as follows.

That is, the specific compound has a structure in which five specific rings are ring-fused at the center as a mother nucleus, and contains aromatic ring groups on both ends of the mother nucleus. Such a specific compound has appropriate crystallinity, and in a case where the photoelectric conversion film containing the specific compound and the n-type semiconductor material is manufactured by vapor deposition, it is easy to keep a crystal state of the entire photoelectric conversion film constant even though a compositional ratio of the manufactured photoelectric conversion film fluctuates. Therefore, it is presumed that the performance of the photoelectric conversion element is stable even though the compositional ratio of the photoelectric conversion film manufactured by vapor deposition fluctuates.

In addition, the photoelectric conversion element including the photoelectric conversion film manufactured by using the specific compound is also excellent in heat resistance. It is believed that this is derived from a rigid structure of the specific compound.

Hereinafter, the performance of the photoelectric conversion element being stabilized even though the compositional ratio of the photoelectric conversion film manufactured by vapor deposition fluctuates (also simply referred to as “excellent in tolerance to composition fluctuation”), and/or the heat resistance of the obtained photoelectric conversion element being excellent (also simply referred to as “excellent in heat resistance”) is also simply referred to as “excellent in the effect of the present invention”.

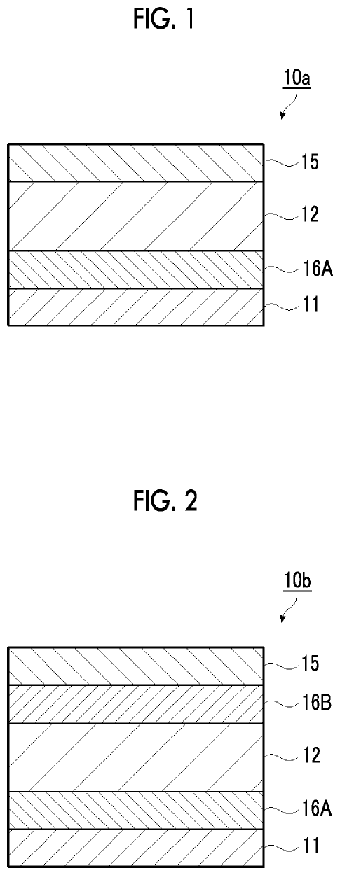

FIG. 1 is a schematic cross-sectional view of one embodiment of a photoelectric conversion element of the present invention.

A photoelectric conversion element 10a shown in FIG. 1 has a configuration in which a conductive film (hereinafter, also referred to as a lower electrode) 11 functioning as a lower electrode, an electron blocking film 16A, a photoelectric conversion film 12 containing the specific compound described later, and a transparent conductive film (hereinafter, also referred to as an upper electrode) 15 functioning as an upper electrode are laminated in this order.

FIG. 2 shows a configuration example of another photoelectric conversion element. A photoelectric conversion element 10b shown in FIG. 2 has a configuration in which the electron blocking film 16A, the photoelectric conversion film 12, a positive hole blocking film 16B, and the upper electrode 15 are laminated on the lower electrode 11 in this order. The lamination order of the electron blocking film 16A, the photoelectric conversion film 12, and the positive hole blocking film 16B in FIGS. 1 and 2 may be appropriately changed according to the application and the characteristics.

In the photoelectric conversion element 10a (or 10b), it is preferable that light is incident on the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage of 1×10⁻⁵ to 1×10⁷ V/cm is applied between the pair of electrodes. From the viewpoint of the performance and power consumption, the applied voltage is more preferably 1×10⁻⁴ to 1×10⁷ V/cm, and even more preferably 1×10⁻³ to 5×10⁶ V/cm.

Regarding a voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the imaging element.

Hereinafter, the form of each layer constituting the photoelectric conversion element according to the embodiment of the present invention will be described in detail.

<Photoelectric Conversion Film>

The photoelectric conversion film is a film containing a specific compound.

Hereinafter, the specific compound will be described in detail.

(Compound (Specific Compound) Represented by Formula (1))

The specific compound is the compound represented by Formula (1).

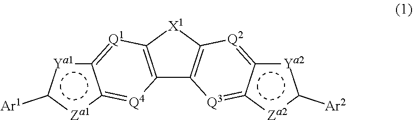

In Formula (1), X¹ represents —O—, —S—, —Se—, —Te—, or —NR^{a1}—.

R^{a1} in —NR^{a1}— represents a hydrogen atom, a halogen atom, an alkyl group which may have a substituent, an alkoxy group which may have a substituent, an alkylthio group which may have a substituent, a silyl group which may have a substituent, an alkenyl group which may have a substituent, an alkynyl group which may have a substituent, an aryl group which may have a substituent, or a heteroaryl group which may have a substituent.

As the alkyl group, alkylthio group, alkoxy group, aryl group, and heteroaryl group, for example, the above-described alkyl group, alkylthio group, alkoxy group, aryl group, and heteroaryl group can be used, respectively.

The alkenyl group may be any of linear, branched, or cyclic. The alkenyl group preferably has 2 to 20 carbon atoms. Examples of the substituent which may be contained in the alkenyl group include the same examples as substituents in the alkyl group which may have a substituent.

The alkynyl group may be any of linear, branched, or cyclic. The alkynyl group preferably has 2 to 20 carbon atoms. Examples of the substituent which may be contained in the alkynyl group include the same examples as substituents in the alkyl group which may have a substituent.

Examples of the silyl group include a group represented by —Si(R^{S1})(R^{S2})(R^{S3}). R^{S1}, R^{S2}, and R^{S3} each independently represent an alkyl group which may have a substituent, an alkoxy group which may have a substituent, an alkylthio group which may have a substituent, an aryl group which may have a substituent, or a heteroaryl group which may have a substituent.

Among these, X¹ is preferably —O—, —S—, or —Se—, and more preferably —S—.

In Formula (1), one of Y^{a1} and Z^{a1} represents —CR^{a2}═ or —N═, and the other represents —O—, —S—, —Se—, —Te—, or —NR^{a3}—.

That is, for example, in a case where Y^{a1} represents —O—, —S—, —Se—, —Te—, or —NR^{a3}—, Z^{a1} represents —CR^{a2}═ or —N═.

In Formula (1), one of Y^{a2} and Z^{a2} represents —CR^{a2}═ or —N═, and the other represents —O—, —S—, —Se—, —Te—, or —NR^{a3}—.

That is, for example, in a case where Y^{a2} represents —O—, —S—, —Se—, —Te—, or —NR^{a3}—, Z^{a2} represents —CR^{a2}═ or —N═.

In —CR^{a2}═ and —NR^{a3}—, R^{a2} and R^{a3} each independently represent a hydrogen atom, a halogen atom, an alkyl group which may have a substituent, an alkoxy group which may have a substituent, an alkylthio group which may have a substituent, a silyl group which may have a substituent, an alkenyl group which may have a substituent, an alkynyl group which may have a substituent, an aryl group which may have a substituent, or a heteroaryl group which may have a substituent.

Examples of R^{a2} and R^{a3} include the same groups described in the explanation of R^{a1}.

In a case where a plurality of R^{a2}'s exist in Formula (1), the plurality of R^{a2}'s may be the same or different from each other.

In a case where a plurality of R^{a3}'s exist in Formula (1), the plurality of R^{a3}'s may be the same or different from each other.

In Formula (1), a 5-membered ring containing Y^{a1} and Z^{a1} is an aromatic heterocycle, and a 5-membered ring containing Y^{a2} and Z^{a2} is an aromatic heterocycle.

Among these, Y^{a1} and Y^{a2} each independently preferably represent —O—, —S—, —Se—, —Te—, or —NR^{a3}—, and Z^{a1} and Z^{a2} each independently preferably represent —CR^{a2}═ or —N═.

Y^{a1} and Y^{a2} each independently preferably represent —O—, —S—, or —Se—, and Z^{a1} and Z^{a2} preferably represent —CR^{a2}═.

It is even more preferable that Y^{a1} and Y^{a2} represent —S—, and Z^{a1} and Z^{a2} represent —CR^{a2}═.

In Formula (1), Q¹ to Q⁴ each independently represent —CR^{a4}═ or —N═.

R^{a4} in —CR^{a4}═ each independently represent a hydrogen atom, a halogen atom, an alkyl group which may have a substituent, an alkoxy group which may have a substituent, an alkylthio group which may have a substituent, a silyl group which may have a substituent, an alkenyl group which may have a substituent, an alkynyl group which may have a substituent, an aryl group which may have a substituent, or a heteroaryl group which may have a substituent.

Examples of R^{a4} include the same groups described in the explanation of R^{a1}.

In a case where a plurality of R^{a4}'s exist in Formula (1), the plurality of R^{a4}'s may be the same or different from each other.

Among these, Q¹ to Q⁴ are each independently preferably —CR^{a4}═, and more preferably —CH═.

In Formula (1), Ar¹ and Ar² each independently represent an aromatic ring group which may have a substituent.

The aromatic ring group may be monocyclic or polycyclic.

The aromatic ring group may or may not contain one or more (preferably 1 to 3) heteroatoms (such as nitrogen atoms, sulfur atoms, oxygen atoms, selenium atoms, tellurium atoms, phosphorus atoms, silicon atoms, and/or boron atoms) as ring member atoms. The number of ring members of the aromatic ring group is preferably 5 to 18.

In a case where the aromatic ring group is a monocyclic aromatic ring group, examples of the monocyclic aromatic ring group include a benzene ring group, a furyl ring group, a pyridine ring group, a pyrazine ring group, a pyrimidine ring group, a pyridazine ring group, a triazine ring group, an oxazole ring group, a thiazole ring group, an imidazole ring group, a pyrazole ring group, an isoxazole ring group, an isothiazole ring group, an oxadiazole ring group, a thiadiazole ring group, a triazole ring group, a tetrazole ring group, a thiophene ring group, a selenophene ring group, and a pyrrole ring group.

A polycyclic aromatic ring group is a group formed by aromatic monocycles being ring-fused with each other. In the polycyclic aromatic ring group, two or more of ring member atoms in each monocycle (aromatic monocycle) constituting a polycyclic aromatic ring are ring member atoms in another monocycle (aromatic monocycle) constituting the polycyclic aromatic ring group.

In a case where the aromatic ring group is a polycyclic aromatic ring group, examples of the polycyclic aromatic ring group include a naphthalene ring group, an anthracene ring group, a quinoline ring group, an isoquinoline ring group, an acridine ring group, a phenanthridine ring group, a pteridine ring group, a quinoxaline ring group, a quinazoline ring group, a cinnoline ring group, a phthalazine ring group, a benzoxazole ring group, a benzothiazole ring group, a benzimidazole ring group, an indazole ring group, a benzoisoxazole ring group, a benzisothiazol ring group, a benzofuran ring group, a benzothiophene ring group, a benzoselenophene ring group, a dibenzofuran ring group, a dibenzothiophene ring group, a dibenzoselenophene ring group, a thienothiophene ring group, a thienopyrrole ring group, a dithienopyrrole ring group, an indole ring group, an imidazopyridine ring group, and a carbazole ring group.

Examples of a substituent which may be contained in the aromatic ring group include the substituent W. Among these, a halogen atom, an alkyl group which may have a substituent, an aryl group which may have a substituent, or a heteroaryl group which may have a substituent is preferable.

In addition, it is also preferable that the aromatic ring group further has an aromatic ring group as a substituent. Examples of the above-described “aromatic ring group as a substituent” include the above-described monocyclic aromatic ring group and polycyclic aromatic ring group.

In addition, in a case where the aromatic ring group further has an aromatic ring group as a substituent, one or more of these aromatic ring groups may have further different substituents, respectively. In addition, the above-described further different substituent contained in each of these aromatic ring groups may be bonded to each other. That is, these aromatic ring groups may be bonded to each other by a further different ring being formed therebetween. However, the further different ring formed between these aromatic ring groups is a non-aromatic ring.

For example, in a case where an aromatic ring group A further has an aromatic ring group B as a substituent, the aromatic ring group A may further have a substituent A, and the aromatic ring group B may further have a substituent B. The substituent A and the substituent B may be bonded to each other to form a further different ring (non-aromatic ring) between the aromatic ring group A and the aromatic ring group B.

Specific examples of the form in which aromatic ring groups are bonded to each other to form a further different ring (non-aromatic ring) therebetween include the form in which these aromatic ring groups together form a fluorene ring group. That is, Ar¹ and Ar² may be, for example, a fluorene ring group (Ar¹ and Ar² may be a fluorene ring group having a substituent, such as a 9,9-dimethylfluorene ring group).

Among these, from the viewpoint that the effect of the present invention is more excellent, it is preferable that Ar¹ and Ar² are each independently a polycyclic aromatic hydrocarbon ring group which may have a substituent or a group represented by Formula (R).

The polycyclic aromatic hydrocarbon ring group may have all ring member atoms as carbon atoms, and a substituent contained in the polycyclic aromatic hydrocarbon ring group may contain a heteroatom.

The number of ring members of the polycyclic aromatic hydrocarbon ring group is preferably 10 to 18.

The polycyclic aromatic hydrocarbon ring group is preferably a naphthalene ring group.

A group represented by Formula (R) is shown below.

—Ar^{X}—Ar^{Y} (R)

In formula (R), Ar^{X} represents a monocyclic aromatic ring group which may have a substituent in addition to Ar^{Y}.

Examples of the monocyclic aromatic ring group are as described above, and among these, a benzene ring group is preferable.

Ar^{X} represents an aromatic ring group which may have a substituent. Examples of the aromatic ring group of Ar^{Y} include the above-described monocyclic aromatic ring group and the above-described polycyclic aromatic ring group. Among these, a benzene ring group or a benzothiazole ring group is preferable.

A monocyclic aromatic ring group in Ar^{X} and an aromatic ring group in Ar^{Y} are bonded to each other by ring member atoms to form a single bond.

Examples of a substituent which may be contained in the monocyclic aromatic ring group in Ar^{X} in addition to Ar^{X} and a substituent which may be contained in the aromatic ring group in Ar^{Y} include the substituent W.

It is also preferable that the monocyclic aromatic ring group in Ar^{X} has no substituent in addition to Ar^{Y}.

The substituent which may be contained in the aromatic ring group in Ar^{Y} is preferably a halogen atom, an alkyl group which may have a substituent, an aryl group which may have a substituent, or a heteroaryl group which may have a substituent.

However, the substituent which may be contained in the monocyclic aromatic ring group in Ar^{X} in addition to Ar^{Y} and the substituent which may be contained in the aromatic ring group in Ar^{Y} are not bonded to each other. That is, Ar^{X} and Ar^{Y} are not bonded except for the single bond specified in Formula (R). For example, the group represented by Formula (R) includes no fluorene ring group.

The specific compound preferably has a symmetrical structure. That is, it is also preferable that Y^{a1} and Y^{a2} are the same, it is also preferable that Z^{a1} and Z^{a2} are the same, it is also preferable that Q¹ and Q² are the same, it is also preferable that Q³ and Q⁴ are the same, and it is also preferable that Ar¹ and Ar² are the same.

(Compound Represented by Formula (2))

From the viewpoint that the effect of the present invention is more excellent, the compound represented by Formula (1) is preferably a compound represented by Formula (2).

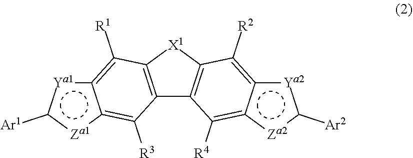

In Formula (2), X¹ represents —O—, —S—, —Se—, —Te—, or —NR^{a1}—. X¹ in Formula (2) is the same as X¹ in Formula (1).

In Formula (2), one of Y^{a1} and Z^{a1} represents —CR^{a2}═ or —N═, and the other represents —O—, —S—, —Se—, —Te—, or —NR^{a3}—. Y^{a1} and Z^{a1} in Formula (2) are the same as Y^{a1} and Z^{a1} in Formula (1).

In Formula (2), one of Y^{a2} and Z^{a2} represents —CR^{a2}═ or —N═, and the other represents —O—, —S—, —Se—, —Te—, or —NR^{a3}—. Y^{a2} and Z^{a2} in Formula (2) are the same as Y^{a2} and Z^{a2} in Formula (1).

In Formula (2), R¹ to R⁴ each independently represent a hydrogen atom, a halogen atom, an alkyl group which may have a substituent, an alkoxy group which may have a substituent, an alkylthio group which may have a substituent, a silyl group which may have a substituent, an alkenyl group which may have a substituent, an alkynyl group which may have a substituent, an aryl group which may have a substituent, or a heteroaryl group which may have a substituent. R¹ to R⁴ in Formula (2) each are the same as R^{a4} in Formula (1). R¹ to R⁴ each are preferably a hydrogen atom.

In Formula (2), Ar¹ and Ar² each independently represent an aromatic ring group which may have a substituent. Ar¹ and Ar² in Formula (2) are the same as Ar¹ and Ar² in Formula (1).

(Compound Represented by Formula (3))

From the viewpoint that the effect of the present invention is more excellent, the compound represented by Formula (1) is more preferably a compound represented by Formula (3).

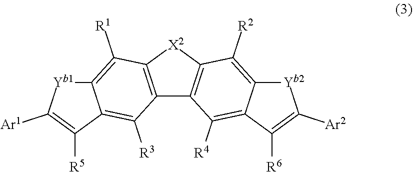

In Formula (3), X² represents —O—, —S—, or —Se—. X² is preferably —S—.

In Formula (3), Y^{b1} and Y^{b2} each independently represent —O—, —S—, or —Se—. Y^{b1} and Y^{b2} each are preferably —S—.

In Formula (3), R¹ to R⁶ each independently represent a hydrogen atom, a halogen atom, an alkyl group which may have a substituent, an alkoxy group which may have a substituent, an alkylthio group which may have a substituent, a silyl group which may have a substituent, an alkenyl group which may have a substituent, an alkynyl group which may have a substituent, an aryl group which may have a substituent, or a heteroaryl group which may have a substituent. R¹ to R⁴ in Formula (3) each are the same as R^{a4} in Formula (1). R¹ to R⁴ each are preferably a hydrogen atom. R⁵ and R⁶ in Formula (4) each are the same as R^{a2} in Formula (1). R⁵ and R⁶ each are preferably a hydrogen atom.

In Formula (3), Ar¹ and Ar² each independently represent an aromatic ring group which may have a substituent. Ar¹ and Ar² in Formula (3) are the same as Ar¹ and Ar² in Formula (1).

Among these, in Formula (3), it is preferable that all of X², Y^{b1}, and Y^{b2} represent —S—.

A molecular weight of the specific compound is not particularly limited, but is preferably 390 to 1200, and more preferably 400 to 900. In a case where the molecular weight is 1200 or less, a vapor deposition temperature is not increased, and the compound is not easily decomposed. In a case where the molecular weight of the compound is 390 or more, a glass transition point of a vapor deposition film is not lowered, and the heat resistance of the photoelectric conversion element is improved.

The specific compound may be used alone, or two or more thereof may be used in combination.

The specific compound is particularly useful as a material of the photoelectric conversion film used for the imaging element, the optical sensor, or a photoelectric cell. The specific compound can also be used as a coloring material, a liquid crystal material, an organic semiconductor material, a charge transport material, a pharmaceutical material, and a fluorescent diagnostic material.

The specific compound is preferably a compound in which an ionization potential in a single film is −5.0 to −6.0 eV from the viewpoints of matching of energy levels between the compound and the n-type semiconductor material described later.

A maximum absorption wavelength of the specific compound is not particularly limited, and for example, is preferably within a range of 300 to 500 nm.

The maximum absorption wavelength is a value measured in a solution state (solvent: chloroform) by an absorption spectrum of the specific compound being adjusted to a concentration having an absorbance of about 0.5 to 1.

A maximum absorption wavelength of the photoelectric conversion film is not particularly limited, and for example, is preferably within a range of 300 to 700 nm.

The specific compounds are exemplified below.

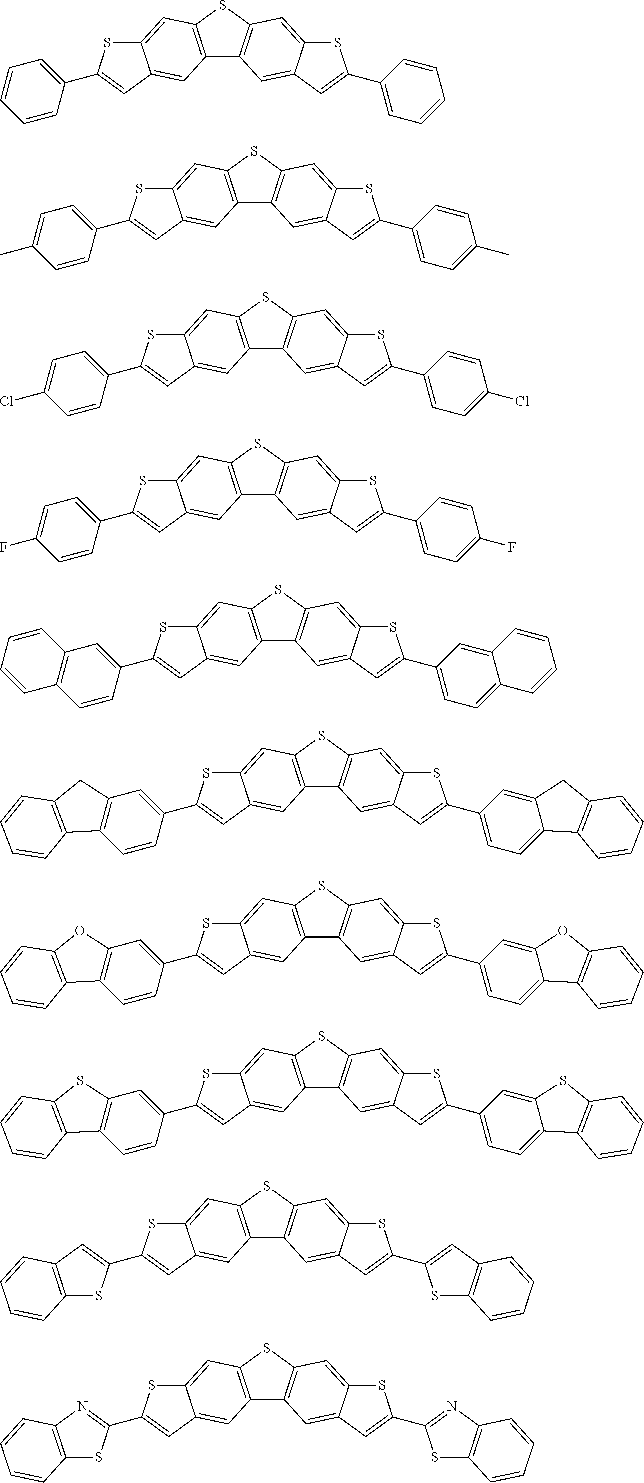

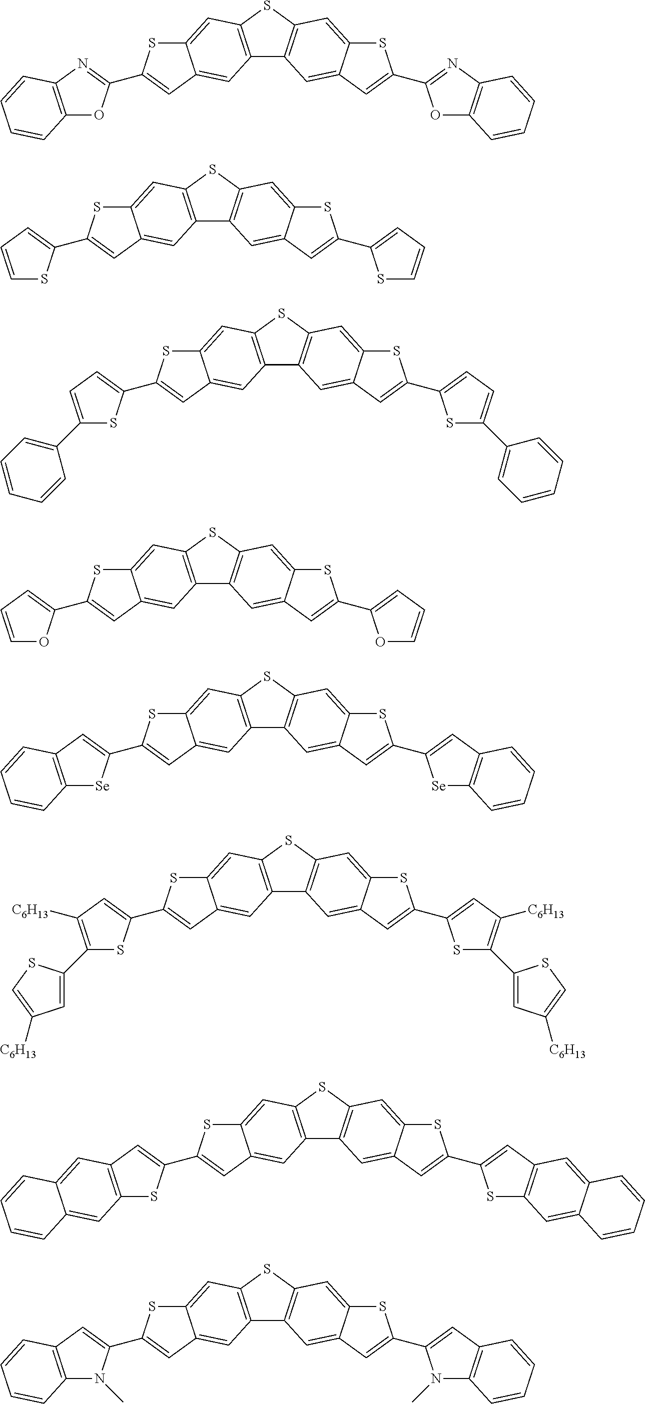

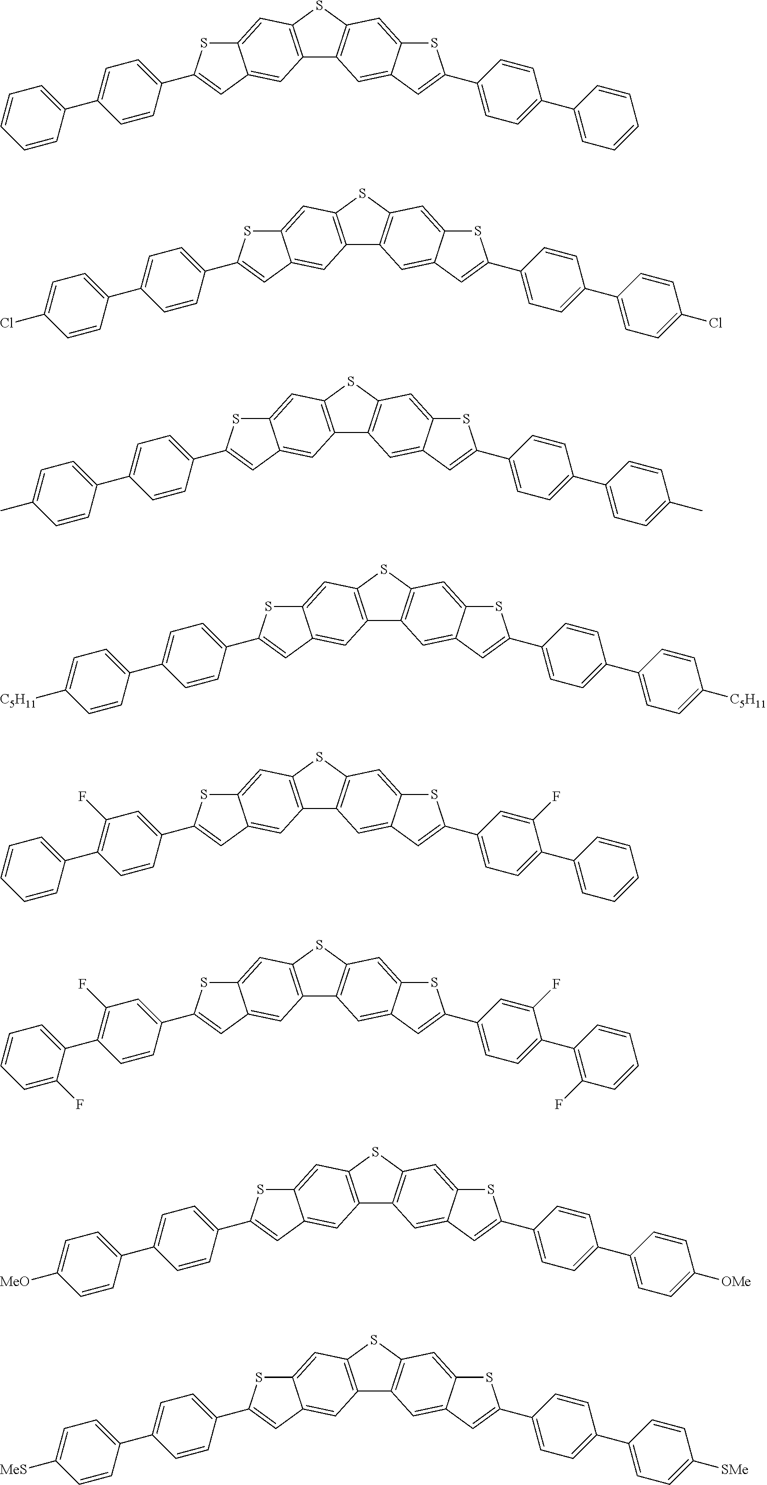

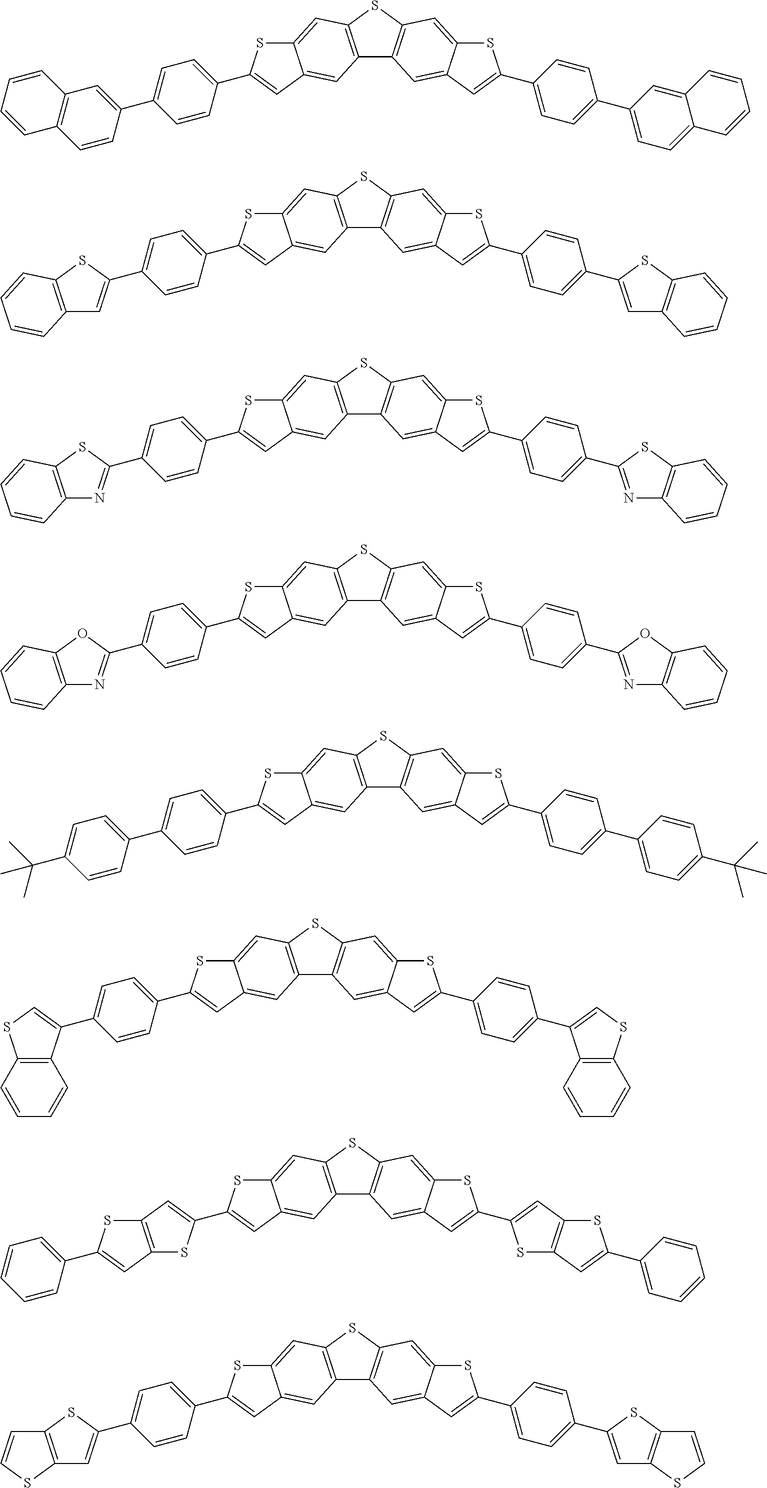

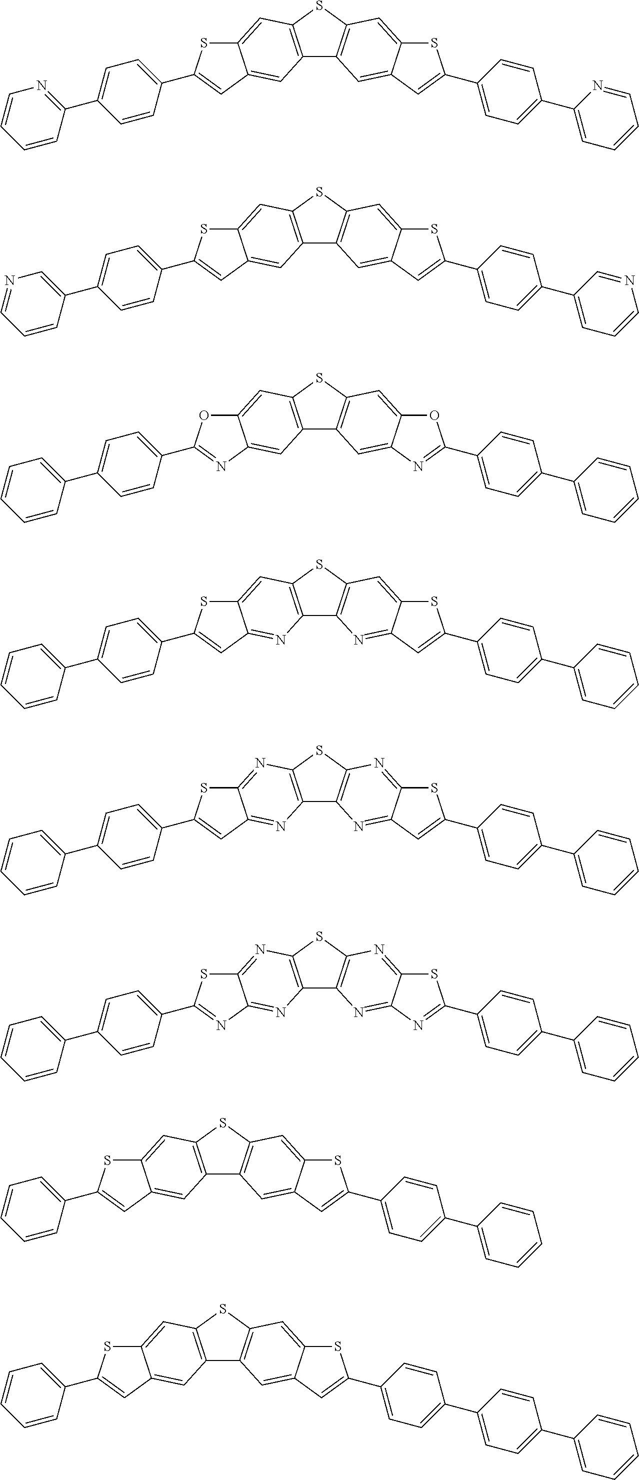

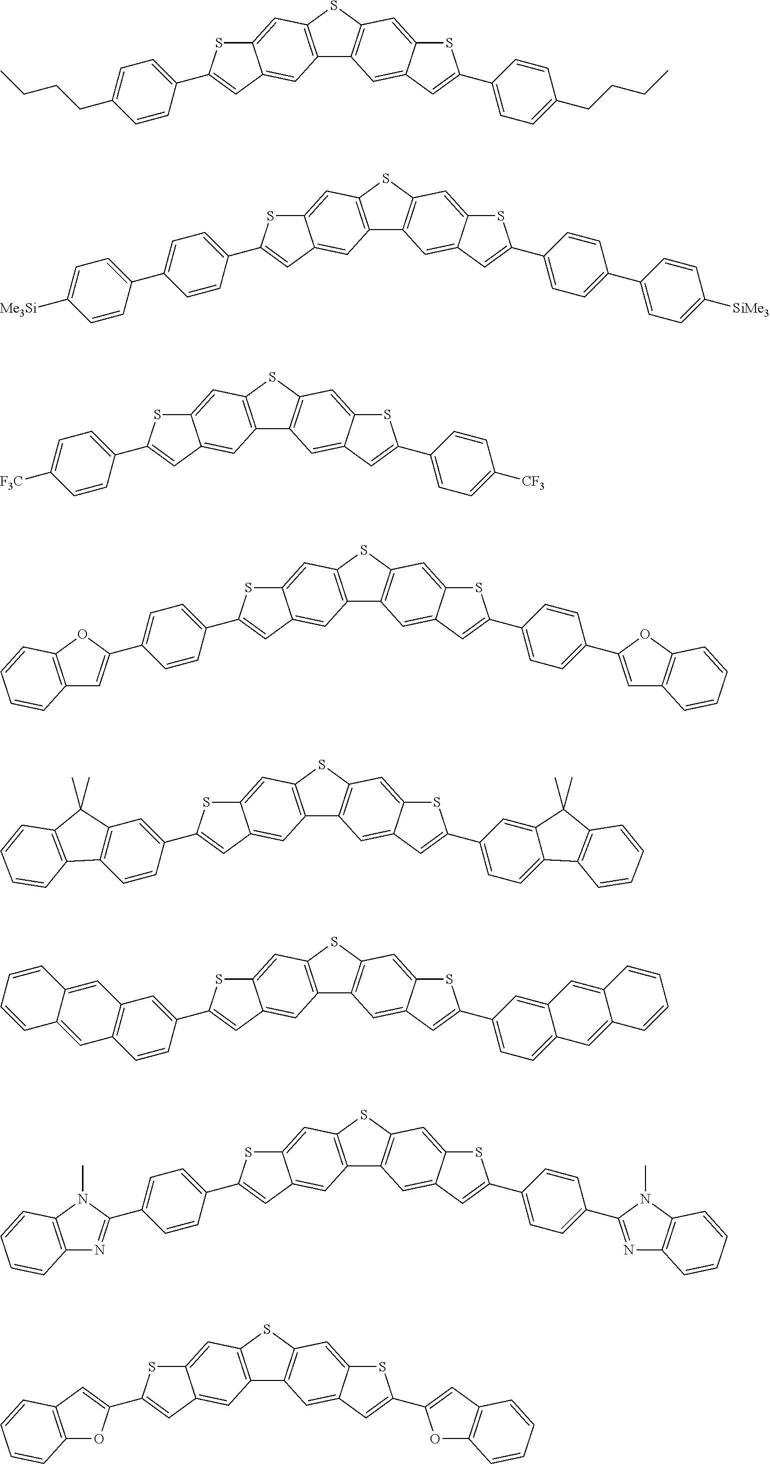

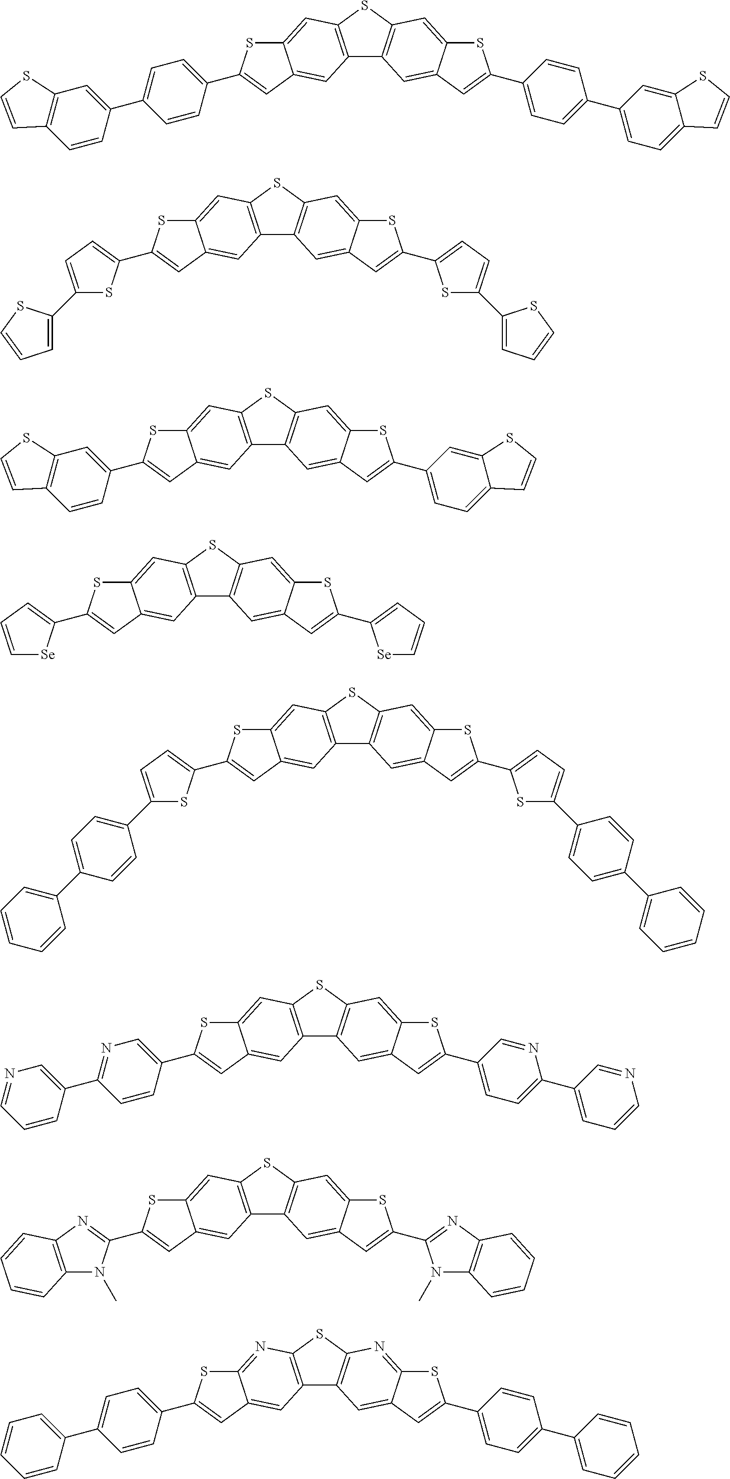

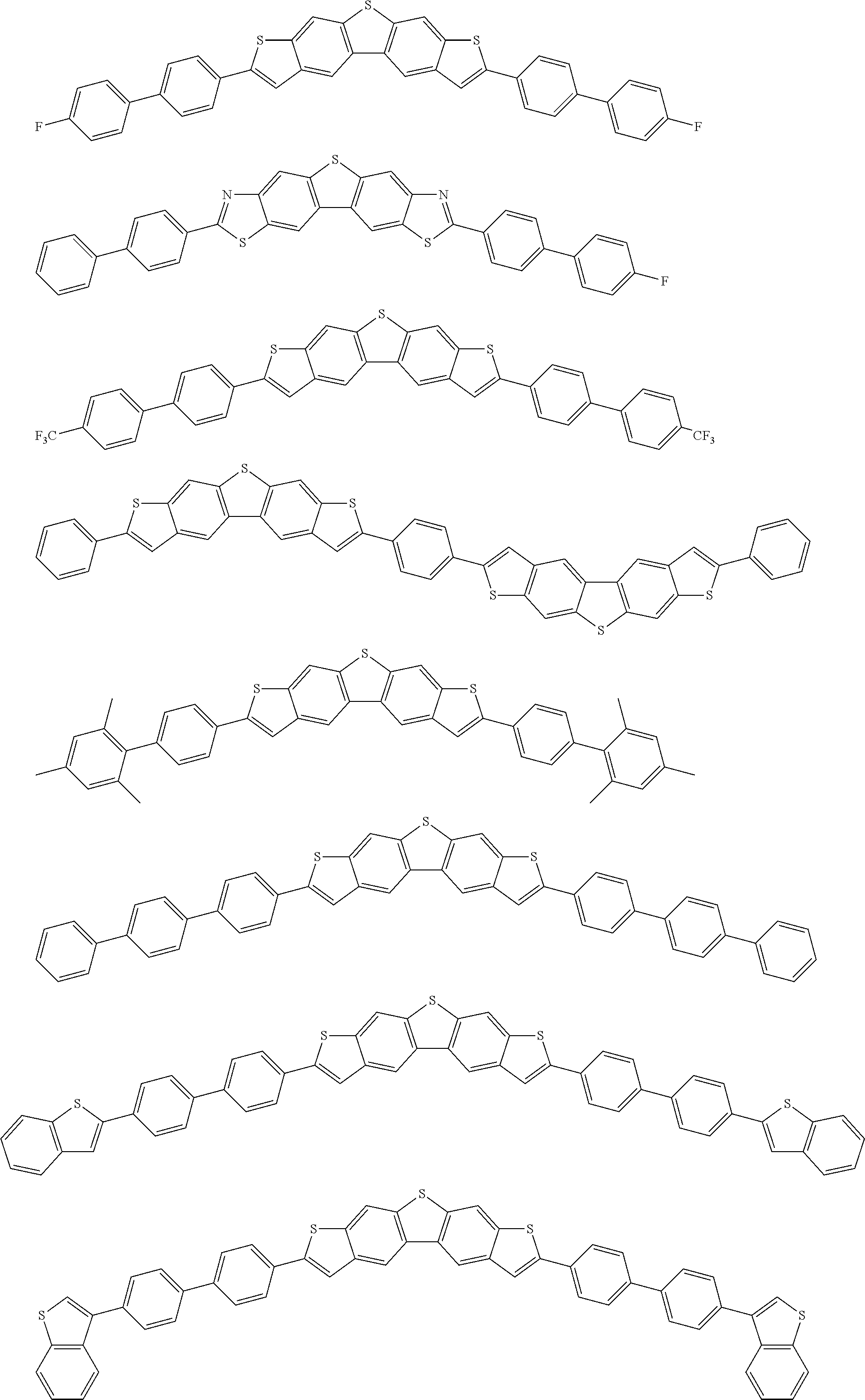

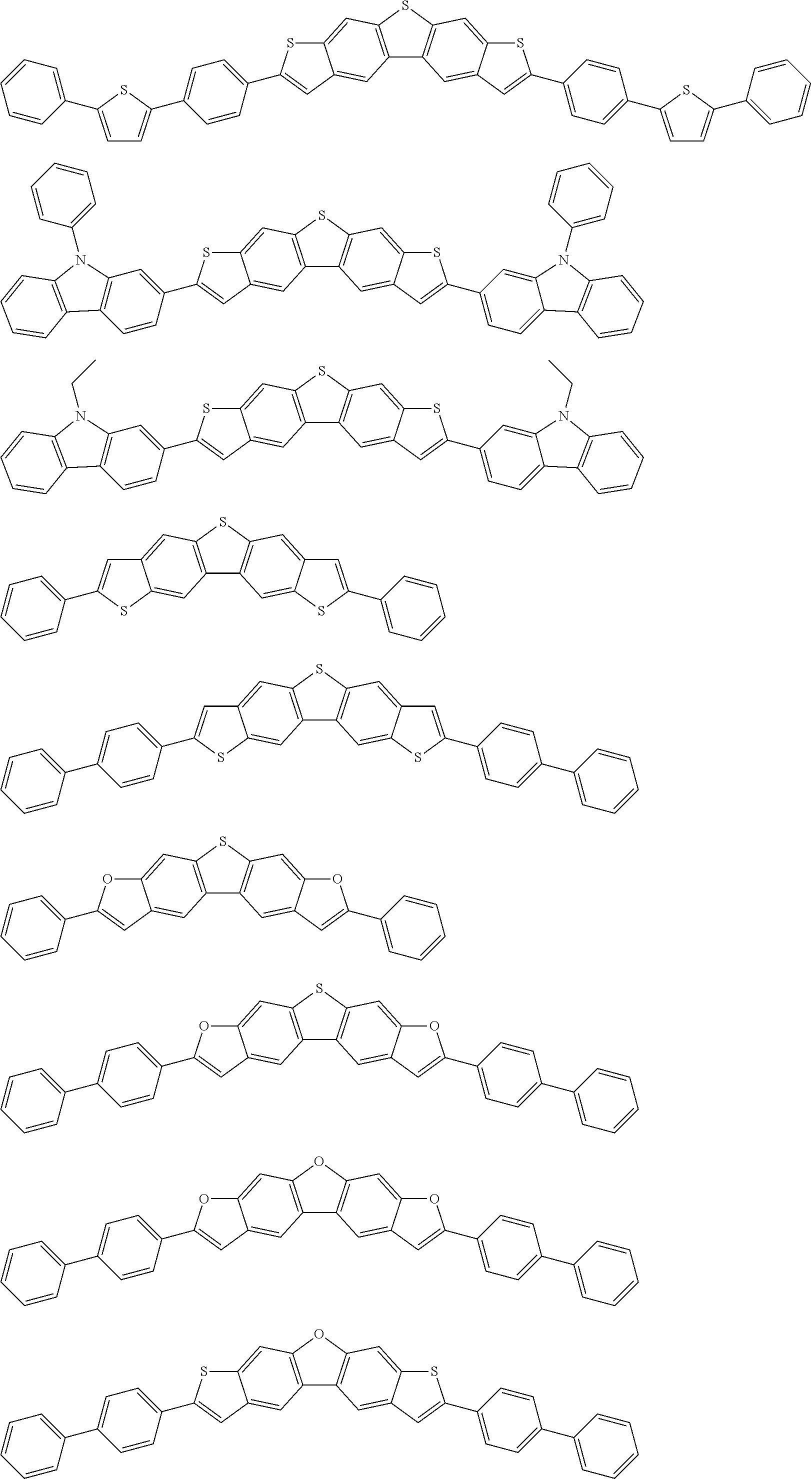

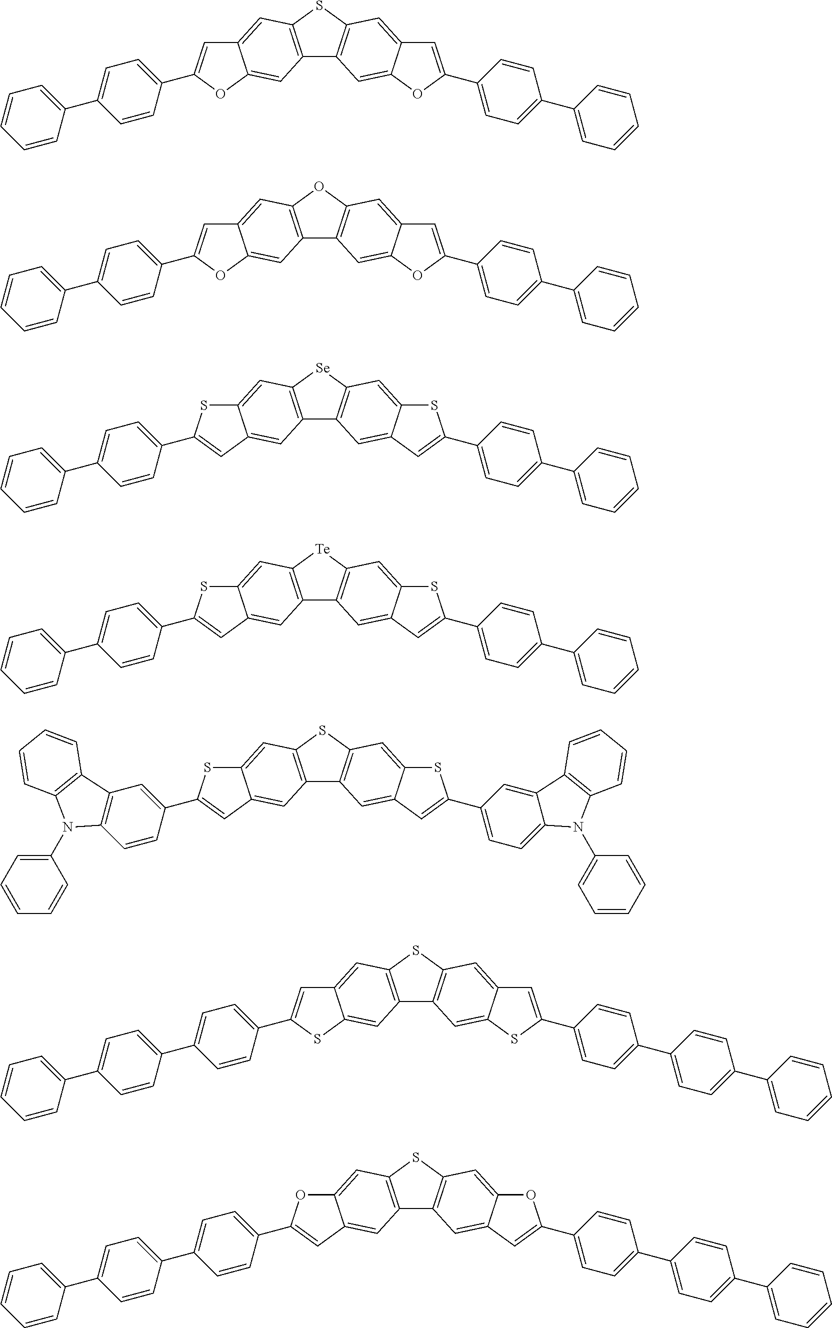

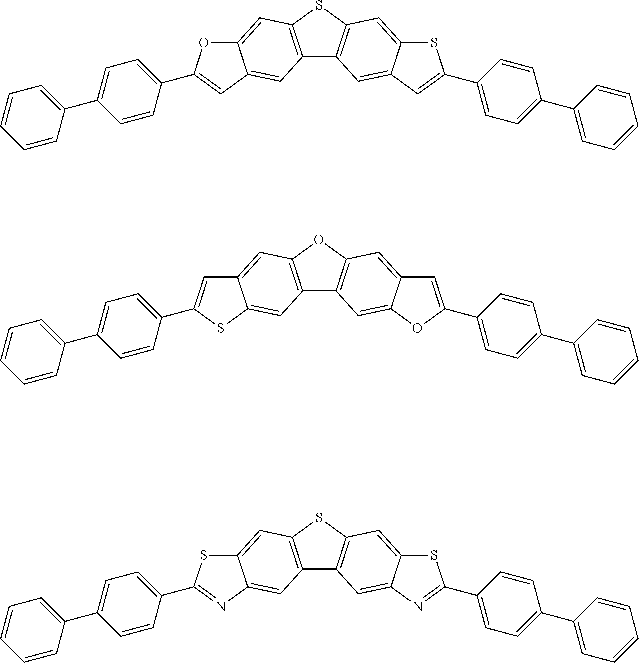 [SMILES: c1ccc(-c2ccc(-c3cc4cc5c(cc4o3)sc3cc4sc(-c6ccc(-c7ccccc7)cc6)cc4cc35)cc2)cc1 ; c1ccc(-c2ccc(-c3cc4cc5oc6cc7cc(-c8ccc(-c9ccccc9)cc8)sc7cc6c5cc4o3)cc2)cc1 ; c1ccc(-c2ccc(-c3nc4cc5c(cc4s3)sc3cc4sc(-c6ccc(-c7ccccc7)cc6)nc4cc35)cc2)cc1]

<N-Type Semiconductor Material>

The photoelectric conversion film contains the n-type semiconductor material as another component in addition to the specific compound. The n-type semiconductor material is an acceptor-property organic semiconductor material (a compound), and refers to an organic compound having a property of easily accepting an electron.

More specifically, the n-type semiconductor material refers to an organic compound having a higher electron affinity than that of the specific compound in a case where the n-type semiconductor material is used by being brought in contact with the above-described specific compound.

In the present specification, a value (value multiplied by −1) of a reciprocal number of the LUMO value obtained by the calculation of B3LYP/6-31G (d) using Gaussian '09 (software manufactured by Gaussian, Inc.) as a value of the electron affinity.

The electron affinity of the n-type semiconductor material is preferably 3.0 to 5.0 eV.

Examples of the n-type semiconductor material include fullerenes selected from the group consisting of a fullerene and derivatives thereof, fused aromatic carbocyclic compounds (for example, a naphthalene derivative, an anthracene derivative, a phenanthrene derivative, a tetracene derivative, a pyrene derivative, a perylene derivative, and a fluoranthene derivative); a heterocyclic compound having a 5- to 7-membered ring having at least one of a nitrogen atom, an oxygen atom, or a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivative; oxadiazole derivative; anthraquinodimethane derivatives; diphenylquinone derivatives; bathocuproine, bathophenanthroline, and derivatives thereof; triazole compounds; a distyrylarylene derivative; a metal complex having a nitrogen-containing heterocyclic compound as a ligand; a silole compound; and compounds disclosed in paragraphs [0056] to [0057] of JP2006-100767A.

Among these, it is preferable that examples of the n-type semiconductor material include fullerenes selected from the group consisting of a fullerene and derivatives thereof.

Examples of the fullerenes include a fullerene C60, a fullerene C70, a fullerene C76, a fullerene C78, a fullerene C80, a fullerene C82, a fullerene C84, a fullerene C90, a fullerene C96, a fullerene C240, a fullerene C540, and a mixed fullerene.

Examples of the fullerene derivatives include compounds in which a substituent is added to the above fullerenes. The substituent is preferably an alkyl group, an aryl group, or a heterocyclic group. The fullerene derivative is preferably compounds described in JP2007-123707A.

In a case where the n-type semiconductor material includes fullerenes, a content of the fullerenes to a total content of the n-type semiconductor material in the photoelectric conversion film (=(film thickness of fullerenes in terms of single layer/film thickness of total n-type semiconductor material in terms of single layer)×100) is preferably 15% to 100% by volume, more preferably 35% to 100% by volume.

An organic coloring agent may be used as the n-type semiconductor material in place of the n-type semiconductor material described in the upper row or together with the n-type semiconductor material described in the upper row.

By using an organic coloring agent as the n-type semiconductor material, it is easy to control an absorption wavelength (maximum absorption wavelength) of the photoelectric conversion element to be within any wavelength range.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, a metal complex coloring agent, compounds disclosed in paragraphs [0083] to [0089] of JP2014-82483A, compounds disclosed in paragraphs [0029] to of JP2009-167348A, compounds disclosed in paragraphs [0197] to [0227] of JP2012-77064A, compounds disclosed in paragraphs [0035] to [0038] of WO2018-105269A, compounds disclosed in paragraphs [0041] to [0043] of WO2018-186389A, compounds disclosed in paragraphs [0059] to [0062] of WO2018-186397A, compounds disclosed in paragraphs [0078] to [0083] of WO2019-009249A, compounds disclosed in paragraphs [0054] to [0056] of WO2019-049946A, compounds disclosed in paragraphs [0059] to [0063] of WO2019-054327A, and compounds disclosed in paragraphs [0086] to [0087] of WO2019-098161A.

In a case where the n-type semiconductor material includes an organic coloring agent, a content of the organic coloring agent to a total content of the n-type semiconductor material in the photoelectric conversion film (=(film thickness of organic coloring agent in terms of single layer/film thickness of total n-type semiconductor material in terms of single layer)×100) is preferably 15% to 100% by volume, more preferably 35% to 100% by volume.

The molecular weight of the n-type semiconductor material is preferably 200 to 1200, and more preferably 200 to 1000.

It is preferable that the photoelectric conversion film has a bulk hetero structure formed in a state where the specific compound and the n-type semiconductor material are mixed. The bulk hetero structure refers to a layer in which the specific compound and the n-type semiconductor material are mixed and dispersed in the photoelectric conversion film. The bulk hetero structure is described in detail in, for example, paragraphs [0013] and [0014] of JP2005-303266A and the like.

From the viewpoint of responsiveness of the photoelectric conversion element, a content of the specific compound to a total content of the specific compound and the n-type semiconductor material (=film thickness of specific compound in terms of a single layer/(film thickness of specific compound in terms of single layer+film thickness of n-type semiconductor material in terms of single layer)×100) is preferably 15% to 75% by volume, and more preferably 35% to 75% by volume.

The photoelectric conversion film is substantially preferably constituted of the specific compound and the n-type semiconductor material. The term “substantially” means that the total content of the specific compound and the n-type semiconductor material is 95% by mass or more with respect to a total mass of the photoelectric conversion film.

The n-type semiconductor material contained in the photoelectric conversion film may be used alone, or two or more thereof may be used in combination.

The photoelectric conversion film containing the specific compound is a non-light emitting film, and has a feature different from an organic light emitting diode (OLED). The non-light emitting film means a film having a light emission quantum efficiency of 1% or less, and the light emission quantum efficiency is preferably 0.5% or less, and more preferably 0.1% or less.

<Film Formation Method>

The photoelectric conversion film can be formed mostly by a dry film formation method. Examples of the dry film formation method include a physical vapor deposition method such as a vapor deposition method (in particular, a vacuum evaporation method), a sputtering method, and an ion plating method, a molecular beam epitaxy (MBE) method, and a chemical vapor deposition (CVD) method such as plasma polymerization. Among these, the vacuum evaporation method is preferable. In a case where the photoelectric conversion film is formed by the vacuum evaporation method, manufacturing conditions such as a degree of vacuum and a vapor deposition temperature can be set according to the normal method.

The thickness of the photoelectric conversion film is preferably 10 to 1000 nm, more preferably 50 to 800 nm, even more preferably 50 to 500 nm, and particularly preferably 50 to 300 nm.

<Electrode>

Electrodes (the upper electrode (the transparent conductive film) 15 and the lower electrode (the conductive film) 11) are formed of conductive materials. Examples of the conductive material include metals, alloys, metal oxides, electrically conductive compounds, and mixtures thereof.

Since light is incident through the upper electrode 15, the upper electrode 15 is preferably transparent to light to be detected. Examples of the materials constituting the upper electrode 15 include conductive metal oxides such as tin oxide (antimony tin oxide (ATO), fluorine doped tin oxide (FTO)) doped with antimony, fluorine, or the like, tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole. Among these, conductive metal oxides are preferable from the viewpoints of high conductivity, transparency, and the like.

In general, in a case where the conductive film is made to be thinner than a certain range, a resistance value is rapidly increased. However, in the solid-state imaging element into which the photoelectric conversion element according to the present embodiment is incorporated, the sheet resistance is preferably 100 to 10000 Ω/□, and a degree of freedom of a range of the film thickness that can be thinned is large. In addition, as the thickness of the upper electrode (the transparent conductive film) 15 is thinner, the amount of light that the upper electrode absorbs is smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the film thickness of the upper electrode 15 is preferably 5 to 100 nm, and more preferably 5 to 20 nm.

There is a case where the lower electrode 11 has transparency or an opposite case where the lower electrode does not have transparency and reflects light, depending on the application. Examples of a material constituting the lower electrode 11 include conductive metal oxides such as tin oxide (ATO, FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum, conductive compounds (for example, titanium nitride (TiN)) such as oxides or nitrides of these metals; mixtures or laminates of these metals and conductive metal oxides; and organic conductive materials such as polyaniline, polythiophene, and polypyrrole.

The method of forming electrodes is not particularly limited, and can be appropriately selected in accordance with the electrode material. Specific examples thereof include a wet method such as a printing method and a coating method; a physical method such as a vacuum evaporation method, a sputtering method, and an ion plating method; and a chemical method such as a CVD method and a plasma CVD method.

In a case where the material of the electrode is ITO, examples thereof include an electron beam method, a sputtering method, a resistance heating vapor deposition method, a chemical reaction method (such as a sol-gel method), and a coating method with a dispersion of indium tin oxide.

<Charge Blocking Film: Electron Blocking Film and Positive Hole Blocking Film>

It is also preferable that the photoelectric conversion element according to the embodiment of the present invention has one or more interlayers between the conductive film and the transparent conductive film, in addition to the photoelectric conversion film. An example of the interlayer includes a charge blocking film. In a case where the photoelectric conversion element has this film, the characteristics (such as photoelectric conversion efficiency and responsiveness) of the obtained photoelectric conversion element are more excellent. Examples of the charge blocking film include an electron blocking film and a positive hole blocking film. Hereinafter, each of the films will be described in detail.

(Electron Blocking Film)

The electron blocking film is a donor organic semiconductor material (compound), and a p-type organic semiconductor described below can be used, for example. The p-type organic semiconductor may be used alone, or two or more thereof may be used in combination.

Examples of the p-type organic semiconductor include triarylamine compounds (for example, N, N′-bis (3-methylphenyl)-(1,1′-biphenyl)-4,4′-diamine (TPD), 4,4′-bis [N-(naphthyl)-N-Phenyl-amino] biphenyl (α-NPD), compounds disclosed in paragraphs [0128] to [0148] of JP2011-228614A, compounds disclosed in paragraphs [0052] to [0063] of JP2011-176259A, compounds disclosed in paragraphs [0119] to [0158] of JP2011-225544A, compounds disclosed in paragraphs [0044] to [0051] of JP2015-153910A, and compounds disclosed in paragraphs [0086] to [0090] of JP2012-94660A, pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1] benzothieno [3,2-b] thiophene (BTBT) derivative, a thieno [3,2-f: 4,5-f′] bis [1] benzothiophene (TBBT) derivative, compounds disclosed in paragraphs [0031] to [0036] of JP2018-14474A, compounds disclosed in paragraphs [0043] to [0045] of WO2016-194630A, compounds disclosed in paragraphs [0025] to [0037], and [0099] to [0109] of WO2017-159684A, compounds disclosed in paragraphs [0029] to [0034] of JP2017-076766A, compounds disclosed in paragraphs [0015] to [0025] of WO2018-207722A, compounds disclosed in paragraphs [0045] to [0053] of JP2019-54228A, compounds disclosed in paragraphs [0045] to [0055] of WO2019-058995A, compounds disclosed in paragraphs [0063] to [0089] of WO2019-081416A, compounds disclosed in paragraphs [0033] to [0036] of JP2019-80052A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, and a fluoranthene derivative), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having nitrogen-containing heterocyclic compounds as ligands.

Examples of the p-type organic semiconductor include compounds having an ionization potential smaller than that of the n-type semiconductor material, and in a case where this condition is satisfied, the organic coloring agents exemplified as the n-type semiconductor material can be used.

A polymer material can also be used as the electron blocking film.

Examples of the polymer material include a polymer such as phenylenevinylene, fluorene, carbazole, indole, pyrene, pyrrole, picoline, thiophene, acetylene, and diacetylene, and a derivative thereof.

The electron blocking film may be formed of a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the photoelectric conversion efficiency increases. Examples of the inorganic material that can be used for the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

(Positive Hole Blocking Film)

A positive hole blocking film is an acceptor-property organic semiconductor material (compound), and the n-type semiconductor material described above can be used.

The method of manufacturing the charge blocking film is not particularly limited, and examples thereof include a dry film formation method and a wet film formation method. Examples of the dry film formation method include a vapor deposition method and a sputtering method. The vapor deposition method may be any of a physical vapor deposition (PVD) method and a chemical vapor deposition (CVD) method, and the physical vapor deposition method such as a vacuum evaporation method is preferable. Examples of the wet film formation method include an ink jet method, a spray method, a nozzle printing method, a spin coating method, a dip coating method, a casting method, a die coating method, a roll coating method, a bar coating method, and a gravure coating method, and an ink jet method is preferable from the viewpoint of high accuracy patterning.

Each thickness of the charge blocking films (the electron blocking film and the positive hole blocking film) is preferably 3 to 200 nm, more preferably 5 to 100 nm, and even more preferably 5 to 30 nm.

<Substrate>

The photoelectric conversion element may further include a substrate. Types of the substrate to be used are not particularly limited, and examples of the substrate include a semiconductor substrate, a glass substrate, and a plastic substrate.

A position of the substrate is not particularly limited, and in general, the conductive film, the photoelectric conversion film, and the transparent conductive film are laminated on the substrate in this order.

<Sealing Layer>

The photoelectric conversion element may further include a sealing layer. The performance of a photoelectric conversion material may deteriorate noticeably due to the presence of deterioration factors such as water molecules. The deterioration can be prevented by coating and sealing the entirety of the photoelectric conversion film with the sealing layer such as diamond-like carbon (DLC) or ceramics such as metal oxide, or metal nitride, and metal nitride oxide which are dense and into which water molecules do not permeate.

The material of the sealing layer may be selected and the sealing layer may be manufactured according to the description in paragraphs [0210] to [0215] of JP2011-082508A.

<Imaging Element>

An example of the application of the photoelectric conversion element includes an imaging element. The imaging element is an element that converts optical information of an image into an electric signal. In general, a plurality of the photoelectric conversion elements are arranged in a matrix on the same plane, and an optical signal is converted into an electric signal in each photoelectric conversion element (pixel) to sequentially output the electric signal to the outside of the imaging element for each pixel. Therefore, each pixel is formed of one or more photoelectric conversion elements and one or more transistors.

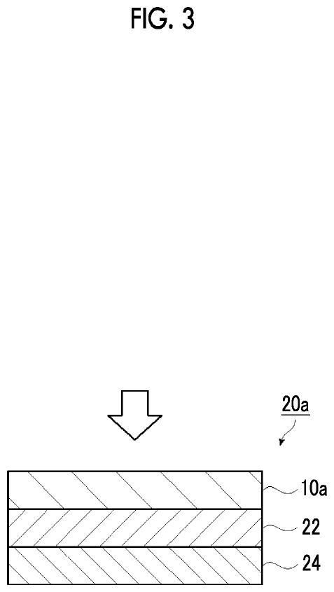

FIG. 3 is a schematic cross-sectional view showing a schematic configuration of an imaging element for describing an embodiment of the present invention. This imaging element is mounted on an imaging element such as a digital camera and a digital video camera, an electronic endoscope, and imaging modules such as a cellular phone.

An imaging element 20a shown in FIG. 3 includes a photoelectric conversion element 10a (a green photoelectric conversion element 10a) according to the embodiment of the present invention, a blue photoelectric conversion element 22, and a red photoelectric conversion element 24, which are laminated along a light incident direction. The photoelectric conversion element 10a is a photoelectric conversion element according to the embodiment of the present invention, and is mostly used as a green photoelectric conversion element by the control of an absorption wavelength so that green light can be received. An example of a method of controlling the absorption wavelength of the photoelectric conversion element according to the embodiment of the present invention includes a method of using an organic coloring agent suitable as the n-type semiconductor material.

The imaging element 20a is a so-called laminated-type color separation imaging element. The photoelectric conversion element 10a, the blue photoelectric conversion element 22, and the red photoelectric conversion element 24 have different wavelength spectra to be detected. That is, the blue photoelectric conversion element 22 and the red photoelectric conversion element 24 correspond to photoelectric conversion elements that receive (absorb) light having a wavelength different from a wavelength of light received by the photoelectric conversion element 10a. The photoelectric conversion element 10a can mostly receive green light, the blue photoelectric conversion element 22 can mostly receive blue light, and the red photoelectric conversion element can mostly receive red light.

Green light means light in a wavelength range of 500 to 600 nm, blue light means light in a wavelength range of 400 to 500 nm, and red light means light in a wavelength range of 600 to 700 nm.

In a case where light is incident on the imaging element 20a in the direction of the arrow, firstly, green light is mostly absorbed by the photoelectric conversion element 10a, but blue light and red light are transmitted through the photoelectric conversion element 10a. In a case where the light transmitted through the photoelectric conversion element 10a travels to the blue photoelectric conversion element 22, the blue light is mostly absorbed, but the red light is transmitted through the blue photoelectric conversion element 22. Thereafter, light transmitted through the blue photoelectric conversion element 22 is absorbed by the red photoelectric conversion element 24. As described above, in the imaging element 20a that is a laminated-type color separation imaging element, one pixel can be formed with three light receiving sections of green, blue, and red, and a large area of the light receiving section can be taken.

The configurations of the blue photoelectric conversion element 22 and the red photoelectric conversion element 24 are not particularly limited.

For example, the photoelectric conversion element having a configuration in which colors are separated by using silicon due to a difference in light absorption length may be used. Further specifically, for example, both the blue photoelectric conversion element 22 and the red photoelectric conversion element 24 may be made of silicon. In this case, as for light including blue light, green light, and red light that has entered the imaging element 20a in the direction of the arrow, the photoelectric conversion element 10a mostly receives the green light having the center wavelength, and the remaining blue light and red light are easily separated. The blue light and red light have different light absorption lengths for silicon (wavelength dependence of absorption coefficient for silicon), the blue light is easily absorbed near a surface of silicon, and the red light can penetrate deeper into the silicon. Based on such a difference in light absorption length, the blue light is mostly received by the blue photoelectric conversion element 22 existing in a shallower position, and the red light is mostly received by the red photoelectric conversion element 24 existing in a deeper position.

In addition, the blue photoelectric conversion element 22 and the red photoelectric conversion element 24 each may be a photoelectric conversion element (the blue photoelectric conversion element 22 or the red photoelectric conversion element 24) having a configuration including a conductive film, an organic photoelectric conversion film having a maximum absorption for blue light or red light, and a transparent conductive film in this order. For example, the blue photoelectric conversion element 22 may be the photoelectric conversion element according to the embodiment of the present invention in which the absorption wavelength is controlled so as to have a maximum absorption for the blue light. Similarly, the red photoelectric conversion element 24 may be the photoelectric conversion element according to the embodiment of the present invention in which the absorption wavelength is controlled so as to have a maximum absorption for the red light.

In FIG. 3, the photoelectric conversion element according to the embodiment of the present invention, the blue photoelectric conversion element, and the red photoelectric conversion element are arranged in this order from the light incident side, but the arrangement is not limited to this aspect, and may be another aspect. For example, the blue photoelectric conversion element, the photoelectric conversion element according to the embodiment of the present invention, and the red photoelectric conversion element may be arranged in this order from the light incident side.

In addition, the green photoelectric conversion element may be used as a photoelectric conversion element other than the photoelectric conversion element according to the embodiment of the present invention, and the blue photoelectric conversion element and/or the red photoelectric conversion element may be used as the photoelectric conversion element according to the embodiment of the present invention.

As described above, the configuration in which the photoelectric conversion elements of the three primary colors of blue, green, and red are laminated as the imaging element is described, but the configuration may be two layers (two colors) or four layers (four colors) or more.

For example, an aspect in which the photoelectric conversion element 10a according to the embodiment of the present invention may be arranged on the arrayed blue photoelectric conversion element 22 and red photoelectric conversion element 24 may be employed. As needed, a color filter that further absorbs light of a predetermined wavelength may be arranged on the light incident side.

The form of the imaging element is not limited to the above-described form and the form shown in FIG. 3 and may be other forms.

For example, an aspect in which the photoelectric conversion element according to the embodiment of the present invention, the blue photoelectric conversion element, and the red photoelectric conversion element may be arranged in the same plane position may be employed.

In addition, the photoelectric conversion element may be used as a single layer. For example, a configuration in which blue, red, and green color filters are arranged on the photoelectric conversion element 10a according to the embodiment of the present invention to separate colors may be employed.

The photoelectric conversion element according to the embodiment of the present invention is also preferably used as an optical sensor. The photoelectric conversion element may be used alone as the optical sensor, and the photoelectric conversion element may be used as a line sensor in which the photoelectric conversion elements are linearly arranged or as a two-dimensional sensor in which the photoelectric conversion elements are arranged in a plane.

<Material for Photoelectric Conversion Element>

The present invention also includes the invention of a material for a photoelectric conversion element.

The material for a photoelectric conversion element according to the embodiment of the present invention is a material used for manufacturing a photoelectric conversion element (preferably a photoelectric conversion element for an imaging element or an optical sensor) and containing a compound (specific compound) represented by Formula (1).

The compound represented by Formula (1) in the material for a photoelectric conversion element is the same as the compound represented by Formula (1) described above, and the preferable conditions are also the same.

It is preferable that each specific compound contained in the material for a photoelectric conversion element is used for producing the photoelectric conversion film contained in the photoelectric conversion element.

Each content of the specific compound contained in the material for a photoelectric conversion element is preferably 30% to 100% by mass, more preferably 70% to 100% by mass, and even more preferably 99% to 100% by mass with respect to the total mass of the material for a photoelectric conversion element.

The specific compound contained in the material for a photoelectric conversion element may be one kind alone or two or more kinds.

### EXAMPLES

The present invention will be described in more detail based on Examples below. Materials, used amounts, ratios, treatment contents, treatment procedures, and the like described in the following Examples can be appropriately changed within the range that does not depart from the gist of the present invention. Therefore, the range of the present invention should not be limitatively interpreted by the following Examples.

<Synthesis of Compound (D-1)>

A compound (D-1) was synthesized according to the following scheme.

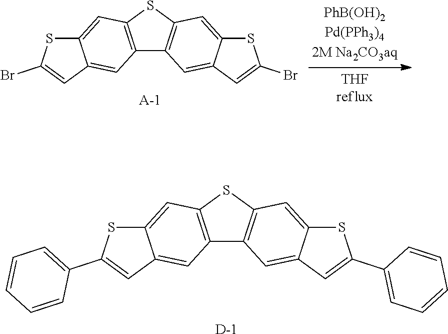

A compound (A-1) was purchased from FUJIFILM Wako Pure Chemical Corporation. Tetrahydrofuran (35 mL) and a 2M aqueous sodium carbonate solution (23 mL) are added to the compound (A-1) (800 mg, 1.76 mmol) and phenylboronic acid (640 mg, 5.28 mmol) to obtain a mixture, and a flask containing the mixture was subjected to nitrogen substitution. Next, nitrogen bubbling was performed on the mixture for five minutes, and furthermore, the dissolved gas in the mixture was degassed under reduced pressure. Thereafter, tetrakis(triphenylphosphine)palladium (0) (47 mg, 0.035 mmol) was added to the above mixture. Thereafter, the mixture was heated and reacted under reflux for seven hours. The mixture after the reaction was allowed to cool and then filtered, and the obtained solid (filtrate) was washed with water and methanol to obtain a crude product. Chlorobenzene (23 mL) was added to the crude product, and dispersion washing was performed at 140° C. for one hour. After washing, the chlorobenzene containing the crude product was allowed to cool and then filtered, and the obtained solid (filtrate) was washed with chlorobenzene and methanol to obtain the compound (D-1) (651 mg, 1.45 mmol, yield 82%).

The obtained compound (D-1) was identified by nuclear magnetic resonance (NMR) and mass spectrometry (MS).

The results of the identification are shown below.

¹H NMR (400 MHz, CDCl₃): δ=7.37 (t,4H), 7.49 (t,2H), 7.71 (s,2H), 7.77 (d,4H), 8.24 (s,2H), 8.59 (s,2H)

MS (MALDI-TOF+) m/z: 449.0 ([M+H]+)

In addition, other specific compounds were further synthesized with reference to the method of synthesizing the compound (D-1).

The compounds (D-1) to (D-15) that are specific compounds, and Compounds (R-1) and (R-2) for comparison are shown below.

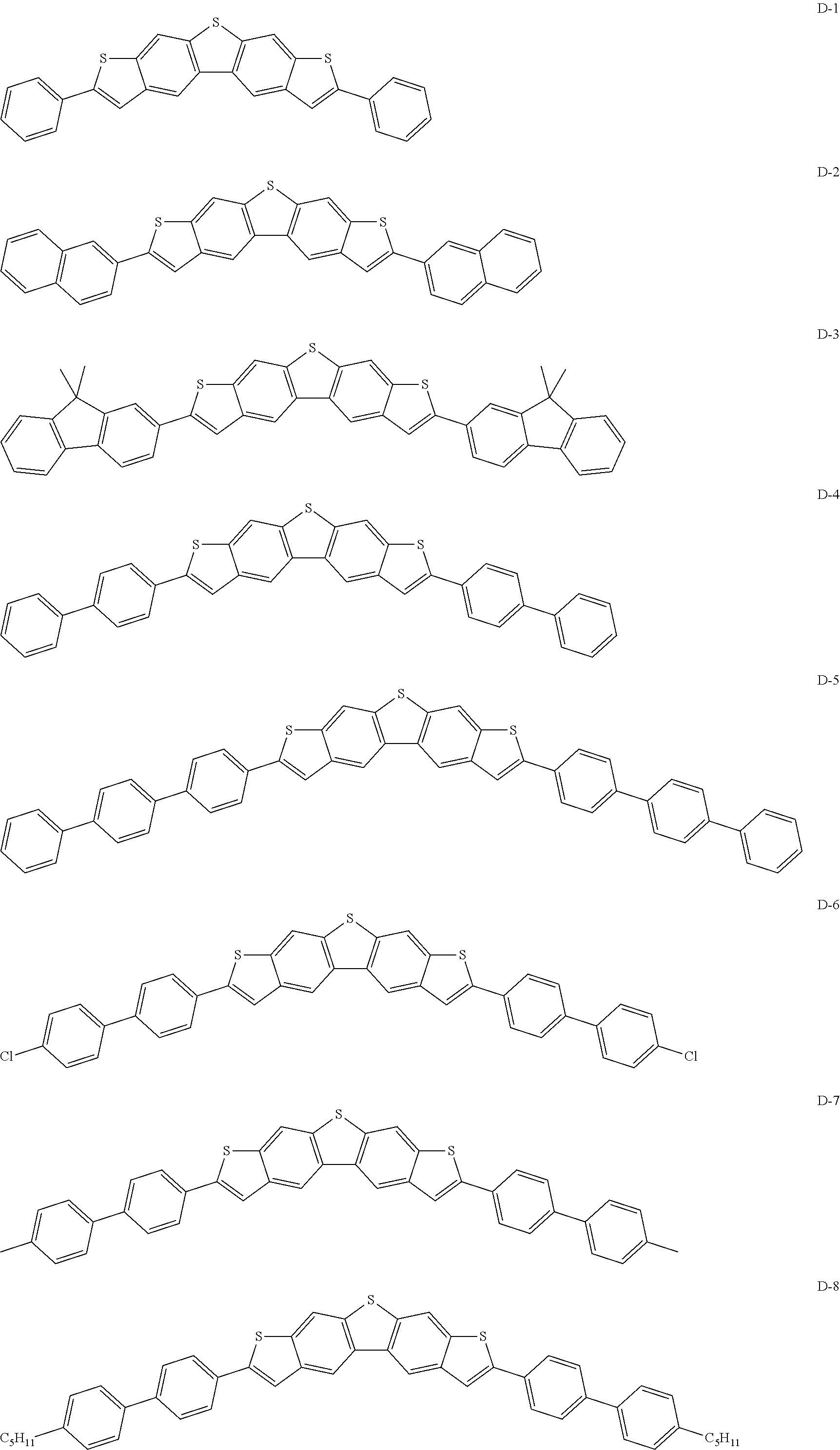

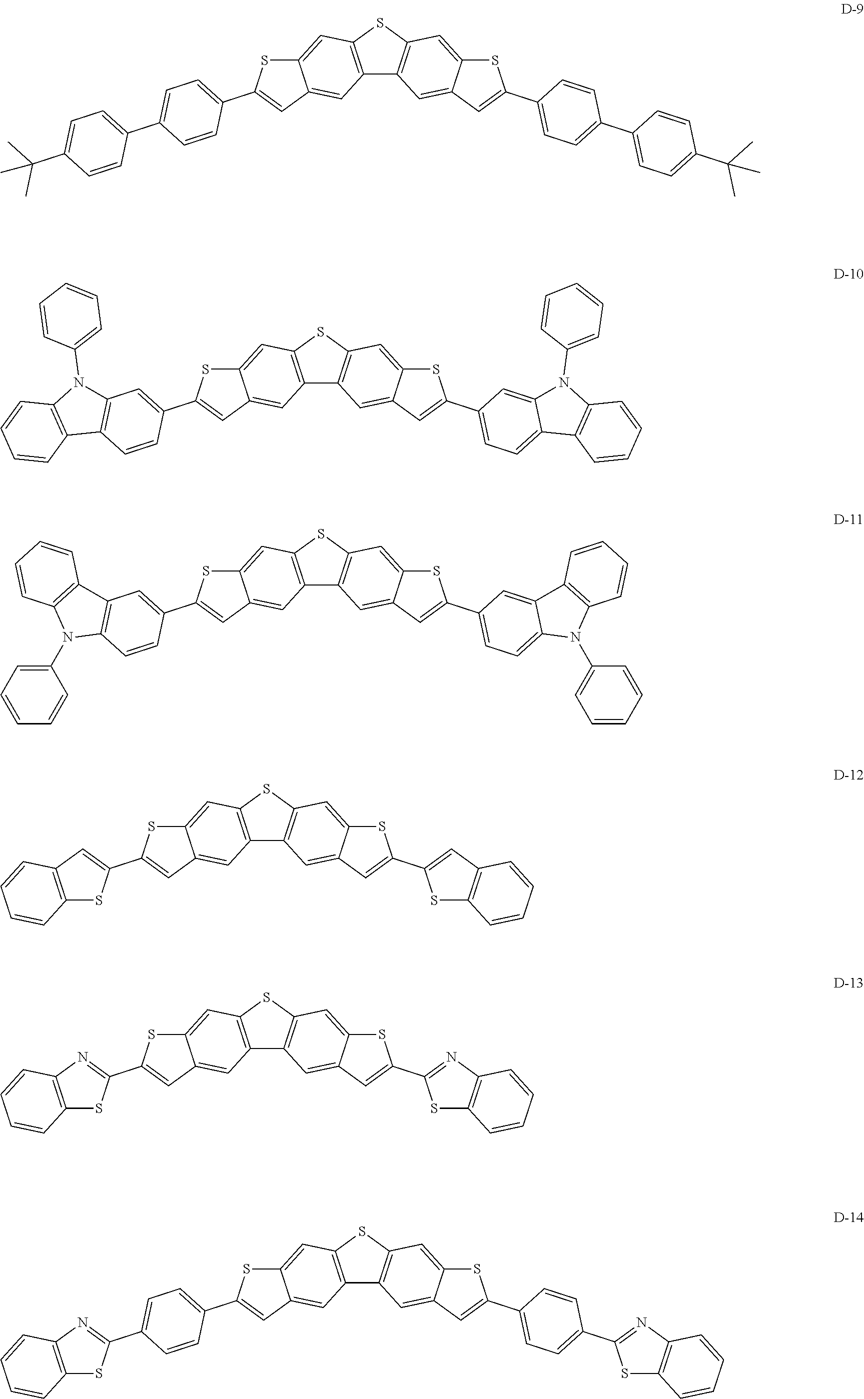

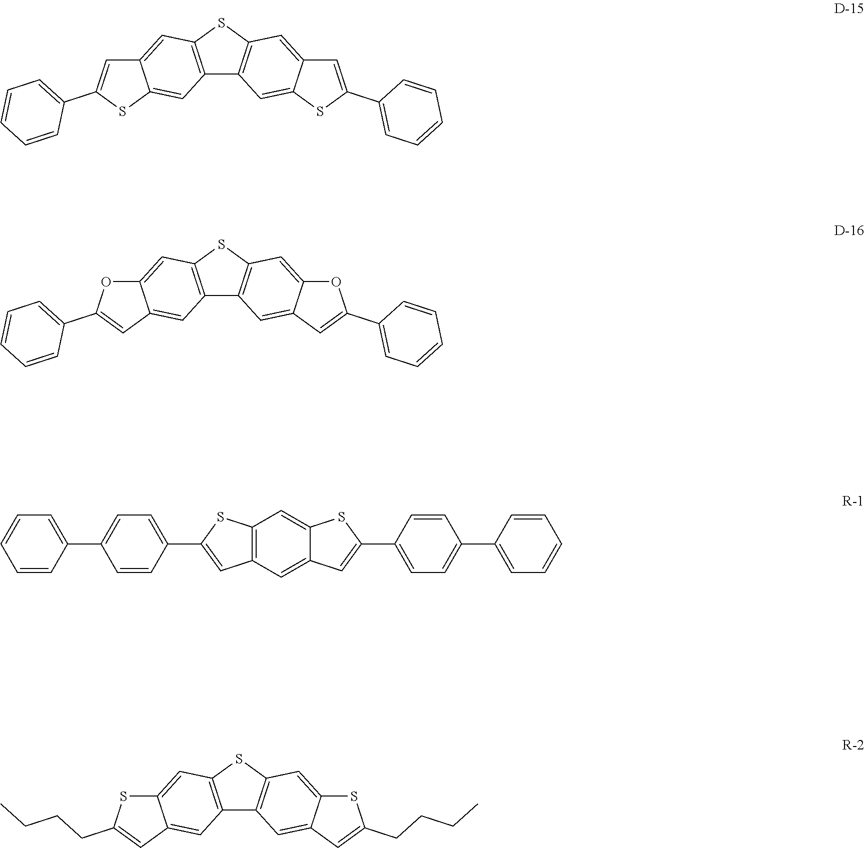 [SMILES: c1ccc(-c2cc3cc4sc5cc6cc(-c7ccccc7)sc6cc5c4cc3s2)cc1 ; c1ccc(-c2cc3cc4c(cc3o2)sc2cc3oc(-c5ccccc5)cc3cc24)cc1 ; c1ccc(-c2ccc(-c3cc4cc5cc(-c6ccc(-c7ccccc7)cc6)sc5cc4s3)cc2)cc1 ; CCCCc1cc2cc3c(cc2s1)sc1cc2sc(CCCC)cc2cc13]

Each of LUMO values of the compounds (D-1) to (D-15), (R-1) and (R-2), and a fullerene (C₆₀) was calculated by B3LYP/6-31G (d) using Gaussian '09 (software manufactured by Gaussian, Inc.). Values of the reciprocal of the obtained LUMO values were adopted as electron affinity values of the compounds.

As a result, it was confirmed that the electron affinity of the fullerene (C₆₀) was larger than the electron affinity of any of the compounds (D-1) to (D-15), and (R-1) and (R-2). That is, it was confirmed that the fullerene (C₆₀) corresponds to an n-type semiconductor material in relation to the compounds (D-1) to (D-15), and (R-1) to (R-2).

<Examples and Comparative Examples: Production of Photoelectric Conversion Element>

The photoelectric conversion element of the form shown in FIG. 2 was produced using the obtained compounds. Here, the photoelectric conversion element includes a lower electrode 11, an electron blocking film 16A, a photoelectric conversion film 12, a positive hole blocking film 16B, and an upper electrode 15.

Specifically, an amorphous ITO was formed into a film on a glass substrate by a sputtering method to form the lower electrode 11 (thickness: 30 nm). Furthermore, a compound (B-1) described below was formed into a film on the lower electrode 11 by a vacuum thermal vapor deposition method to form the electron blocking film 16A (thickness: 10 nm).

Furthermore, the compound (D-1) and the fullerene (C₆₀) were set to a vapor deposition rate of 2.0 Å/sec and subjected to co-vapor deposition by a vacuum evaporation method to be 100 nm and 100 nm respectively, in terms of a single layer, on the electron blocking film 16A to be formed into a film in a state where the temperature of the substrate was controlled to 25° C., and the photoelectric conversion film 12 having a bulk hetero structure of 200 nm was formed (a step of forming a photoelectric conversion film).

Furthermore, a compound (B-2) described below was formed into a film on the photoelectric conversion film 12 to form the positive hole blocking film 16B (thickness: 10 nm).

Furthermore, amorphous ITO was formed into a film on the positive hole blocking film 16B by a sputtering method to form the upper electrode 15 (the transparent conductive film) (thickness: 10 nm). A SiO film was formed as a sealing layer on the upper electrode 15 by a vacuum evaporation method, and thereafter, an aluminum oxide (Al₂O₃) layer was formed thereon by an atomic layer chemical vapor deposition (ALCVD) method to produce a photoelectric conversion element, and this element was denoted by an element (A_{D-1}).

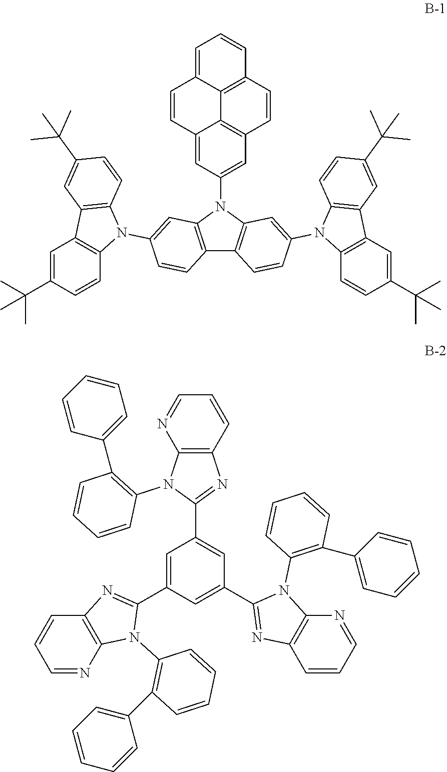 [SMILES: CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3cc4ccc5cccc6ccc(c3)c4c56)c2c1 ; c1ccc(-c2ccccc2-n2c(-c3cc(-c4nc5cccnc5n4-c4ccccc4-c4ccccc4)cc(-c4nc5cccnc5n4-c4ccccc4-c4ccccc4)c3)nc3cccnc32)cc1]

By using each of the compounds (D-2) to (D-16), or (R-1) and (R-2) instead of the compound (D-1), photoelectric conversion elements were produced in the same manner to obtain elements (A_{D-2}) to (A_{D-16}), and (A_{R-1}) and (A_{R-2}).

<Confirmation of Drive (Photoelectric Conversion Efficiency and Evaluation of Dark Current)>

The drive of each of the obtained elements was confirmed. A voltage was applied to have an electric field strength of 1.0×10⁵ V/cm for each element (elements (A_{D-1}) to (A_{D-16}), and (A_{R-1}) and (A_{R-2})). Thereafter, light was emitted from the upper electrode (transparent conductive film) side to evaluate the photoelectric conversion efficiency (external quantum efficiency) at 400 nm. The external quantum efficiency was measured using a constant energy quantum efficiency measuring device manufactured by Optel Co., Ltd. The amount of light emitted was 50 μW/cm². It was confirmed that all the elements showed a photoelectric conversion efficiency of 30% or more and a dark current of 10 nA/cm² or less, and could be driven without any problem.

<Evaluation of Heat Resistance>

Each of the obtained elements (elements (A_{D-1}) to (A_{D-16}), and (A_{R-1}) and (A_{R-2})) was placed in a glove box at 160° C., and heat treated for two hours. Thereafter, the dark current was evaluated by the same method as the above-described <Confirmation of Drive (Photoelectric Conversion Efficiency and Evaluation of Dark Current)>. The evaluation was performed using a relative value in a case where a dark current value before heat treatment was set to 1, and a case where the relative value was 2 or less was evaluated as A, a case where the relative value was more than 2 and 5 or less was evaluated as B, a case where the relative value was more than 5 and 10 or less was evaluated as C, and a case where the relative value was more than 10 was evaluated as D. In practice, A and B are preferable, and A is more preferable. The results are shown in Table 1.

<Tolerance to Composition Fluctuation>

(Production of Photoelectric Conversion Element (Element (B)))

In the production of the element (A_{D-1}), the vapor deposition rates of the compound (D-1) and the fullerene (C₆₀) were set to 2.4 Å/sec and 1.6 Å/sec, respectively, and the photoelectric conversion film 12 having a bulk hetero structure of 200 nm was formed by co-vapor deposition to be formed into a film by a vacuum evaporation method so as to be 120 nm and 80 nm respectively, in terms of a single layer. The photoelectric conversion element was produced such that all other producing conditions were the same as the element (A_{D-1}), and this element was denoted by an element (B_{D-1}).

By using each of the compounds (D-2) to (D-16), or (R-1) and (R-2) instead of the compound (D-1), photoelectric conversion elements were produced in the same manner to obtain elements (B_{D-2}) to (B_{D-16}), and (B_{R-1}) and (B_{R-2}).

(Evaluation of Dark Current)

A voltage was applied to the obtained element (B_{D-1}) to have an electric field strength of 1.0×10⁵ V/cm, the dark current was evaluated, and a tolerance to fluctuation of a compositional ratio was evaluated by a relative value with respect to the value of the element (A_{D-1}).

That is, the relative value was obtained as “relative value=dark current value of element (B_{D-1})/dark current value of element (A_{D-1})”.

Similar for the elements (A_{D-2}) to (A_{D-16}) and (A_{R-1}) and (A_{R-2}), and the elements (B_{D-2}) to (B_{D-16}) and (B_{R-1}) and (B_{R-2}), relative values in a case of using each compound were obtained.

It was evaluated that the closer the relative value of 1, the better the tolerance for fluctuation of the compositional ratio. Specifically, a case where the relative value is more than 0.8 and 1.25 or less was evaluated as A, a case where the relative value is more than 0.5 and 0.8 or less or more than 1.25 and 2.0 or less was evaluated as B, a case where the relative value is more than 0.1 and 0.5 or less or more than 2.0 and 5.0 or less was evaluated as C, and a case where the relative value is 0.1 or less or more than 5.0 was evaluated as D. In practice, A and B are preferable, and A is more preferable. The results are shown in Table 1.

The results of the tests conducted using the photoelectric conversion elements produced by using each compound are shown in Table 1 below.

In Table 1, the column “Formula (3)” indicates whether or not the specific compound used corresponds to the compound represented by Formula (3). A case of satisfying this requirement was evaluated as A, and a case of satisfying no requirement was evaluated as B.

The “Ar¹, Ar²=polycyclic aromatic hydrocarbon/Formula (R)” column indicates whether or not a group corresponding to a group represented by Ar¹, Ar² in Formula (1) in the specific compound is a polycyclic aromatic hydrocarbon ring group which may have a substituent or a group represented by Formula (R). A case of satisfying this requirement was evaluated as A, and a case of satisfying no requirement was evaluated as B.

TABLE 1

| | | Feature of | | | |
|---|---|---|---|---|---|
| | | specific compound | | Evaluation | |
| | | | Ar¹, Ar² = | | Tolerance to |
| | | | polycyclic | | fluctuation |
| | | | aromatic | Heat | of com- |
| | Com- | Formula | hydrocarbon/ | resist- | positional |
| | pound | (3) | Formula (R) | ance | ratio |
| Example 1 | D-1 | A | B | B | A |
| Example 2 | D-2 | A | A | A | A |
| Example 3 | D-3 | A | B | A | B |
| Example 4 | D-4 | A | A | A | A |
| Example 5 | D-5 | A | A | A | A |
| Example 6 | D-6 | A | A | A | A |
| Example 7 | D-7 | A | A | A | A |
| Example 8 | D-8 | A | A | A | A |
| Example 9 | D-9 | A | A | A | A |
| Example 10 | D-10 | A | B | A | B |
| Example 11 | D-11 | A | B | A | B |
| Example 12 | D-12 | A | B | B | A |
| Example 13 | D-13 | A | B | B | A |
| Example 14 | D-14 | A | A | A | A |
| Example 15 | D-15 | B | B | B | B |
| Example 16 | D-16 | A | B | B | A |
| Comparative | R-1 | — | — | D | C |
| Example 1 | | | | | |
| Comparative | R-2 | — | — | C | D |
| Example 2 | | | | | |

From the results shown in Table 1, it was confirmed that the photoelectric conversion element according to the embodiment of the present invention using the specific compound for the photoelectric conversion film exhibits stable performance even though the compositional ratio of the photoelectric conversion film fluctuates in the case where the photoelectric conversion film is manufactured by vapor deposition. In addition, it was confirmed that the photoelectric conversion element according to the embodiment of the present invention has excellent heat resistance.

On the other hand, in the case where the compound (R-1) having the mother nucleus with the structure different from the specific compound was used, the tolerance to fluctuation of the compositional ratio of the photoelectric conversion film was insufficient. In addition, the heat resistance also deteriorated as compared with the photoelectric conversion element according to the embodiment of the present invention.

In addition, in the case where the compound (R-2) having the same mother nucleus as the specific compound but having an alkyl group instead of an aromatic ring group as a group bonded to the mother nucleus was used, the tolerance to fluctuation of the compositional ratio of the photoelectric conversion film was insufficient. In addition, the heat resistance also deteriorated as compared with the photoelectric conversion element according to the embodiment of the present invention.

In addition, it was confirmed that the effect of the present invention is more excellent in the case where the specific compound corresponds to the compound represented by Formula (3) (see the results of comparison between Example 16 and other Examples).

It was confirmed that the effect of the present invention is more excellent in the case where the groups corresponding to Ar¹ and Ar² in Formula (1) of the specific compound are a polycyclic aromatic hydrocarbon ring group which may have a substituent or a group represented by Formula (R) (see the results of comparison between Examples using the specific compound corresponding to the compound represented by Formula (3)).

### EXPLANATION OF REFERENCES

10a and 10b photoelectric conversion element

11 conductive film (lower electrode)

12 photoelectric conversion film

15 transparent conductive film (upper electrode)

16A electron blocking film

16B positive hole blocking film

20a imaging element

22 blue photoelectric conversion element

24 red photoelectric conversion element

What is claimed is:

1. A photoelectric conversion element comprising, in the following order:
a conductive film;
a photoelectric conversion film; and
a transparent conductive film,
wherein the photoelectric conversion film contains a compound represented by Formula (1) and an n-type semiconductor material, 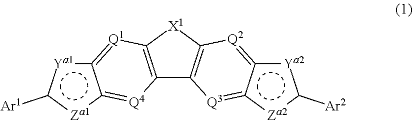
in Formula (1), X¹ represents —O—, —S—, —Se—, —Te—, or —NR^{a1}—,
one of Y^{a1} and Z^{a1} represents —CR^{a2}═ or —N═, and the other represents —O—, —S—, —Se—, —Te—, or —NR^{a3}—,
one of Y^{a2} and Z^{a2} represents —CR^{a2}═ or —N═, and the other represents —O—, —S—, —Se—, —Te—, or —NR^{a3}—,
Q¹ to Q⁴ each independently represent —CR^{a4}═ or —N═,
R^{a1} to R^{a4} each independently represent a hydrogen atom, a halogen atom, an alkyl group which may have a substituent, an alkoxy group which may have a substituent, an alkylthio group which may have a substituent, a silyl group which may have a substituent, an alkenyl group which may have a substituent, an alkynyl group which may have a substituent, an aryl group which may have a substituent, or a heteroaryl group which may have a substituent, and
Ar¹ and Ar² each independently represent an aromatic ring group which may have a substituent.

2. The photoelectric conversion element according to claim 1, wherein the compound represented by Formula (1) is a compound represented by Formula (2), 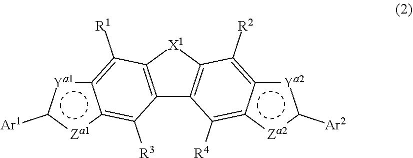
in Formula (2), X¹ represents —O—, —S—, —Se—, —Te—, or —NR^{a1}—,
one of Y^{a1} and Z^{a1} represents —CR^{a2}═ or —N═, and the other represents —O—, —S—, —Se—, —Te—, or —NR^{a3}—,
one of Y^{a2} and Z^{a2} represents —CR^{a2}═ or —N═, and the other represents —O—, —S—, —Se—, —Te—, or —NR^{a3}—,
R^{a1} to R^{a3} and R¹ to R⁴ each independently represent a hydrogen atom, a halogen atom, an alkyl group which may have a substituent, an alkoxy group which may have a substituent, an alkylthio group which may have a substituent, a silyl group which may have a substituent, an alkenyl group which may have a substituent, an alkynyl group which may have a substituent, an aryl group which may have a substituent, or a heteroaryl group which may have a substituent, and
Ar¹ and Ar² each independently represent an aromatic ring group which may have a substituent.

3. The photoelectric conversion element according to claim 1, wherein the compound represented by Formula (1) is a compound represented by Formula (3), 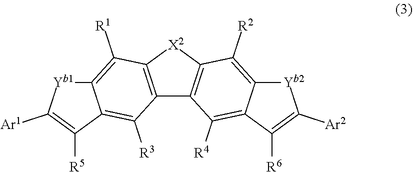
in Formula (3), X² represents —O—, —S—, or —Se—,
Y^{b1} and Y^{b2} each independently represent —O—, —S—, or —Se—,
R¹ to R⁶ each independently represent a hydrogen atom, a halogen atom, an alkyl group which may have a substituent, an alkoxy group which may have a substituent, an alkylthio group which may have a substituent, a silyl group which may have a substituent, an alkenyl group which may have a substituent, an alkynyl group which may have a substituent, an aryl group which may have a substituent, or a heteroaryl group which may have a substituent, and
Ar¹ and Ar² each independently represent an aromatic ring group which may have a substituent.

4. The photoelectric conversion element according to claim 3, wherein X², Y^{b1}, and Y^{b2} represent —S—.

5. The photoelectric conversion element according to claim 1, wherein Ar¹ and Ar² each independently represent a polycyclic aromatic hydrocarbon ring group which may have a substituent or a group represented by Formula (R),
—Ar^{X}—Ar^{Y} (R)
in formula (R), Ar^{X} represents a monocyclic aromatic ring group which may have a substituent in addition to Ar^{Y}, and
Ar^{Y} represents an aromatic ring group which may have a substituent.

6. The photoelectric conversion element according to claim 1, wherein the compound represented by Formula (1) has a molecular weight of 400 to 900.

7. The photoelectric conversion element according to claim 1, wherein the photoelectric conversion film has a bulk hetero structure formed in a state where the compound represented by Formula (1) and the n-type semiconductor material are mixed to each other.

8. The photoelectric conversion element according to claim 1, further comprising one or more interlayers between the conductive film and the transparent conductive film, in addition to the photoelectric conversion film.

9. The photoelectric conversion element according to claim 1, wherein the n-type semiconductor material includes fullerenes selected from the group consisting of a fullerene and a derivative thereof.

10. An imaging element comprising the photoelectric conversion element according to claim 1.

11. An optical sensor comprising the photoelectric conversion element according to claim 1.

12. A material for a photoelectric conversion element comprising a compound represented by Formula (1), 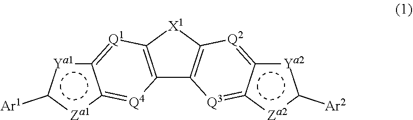
in Formula (1), X¹ represents —O—, —S—, —Se—, —Te—, or —NR^{a1}—,
one of Y^{a1} and Z^{a1} represents —CR^{a2}═ or —N═, and the other represents —O—, —S—, —Se—, —Te—, or —NR^{a3}—,
one of Y^{a2} and Z^{a2} represents —CR^{a2}═ or —N═, and the other represents —O—, —S—, —Se—, —Te—, or —NR^{a3}—,
Q¹ to Q⁴ each independently represent —CR^{a4}═ or —N═,
R^{a1} to R^{a4} each independently represent a hydrogen atom, a halogen atom, an alkyl group which may have a substituent, an alkoxy group which may have a substituent, an alkylthio group which may have a substituent, a silyl group which may have a substituent, an alkenyl group which may have a substituent, an alkynyl group which may have a substituent, an aryl group which may have a substituent, or a heteroaryl group which may have a substituent, and
Ar¹ and Ar² each independently represent an aromatic ring group which may have a substituent.

13. The photoelectric conversion element according to claim 2, wherein the compound represented by Formula (1) is a compound represented by Formula (3), 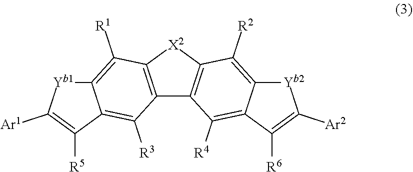
in Formula (3), X² represents —O—, —S—, or —Se—,
Y^{b1} and Y^{b2} each independently represent —O—, —S—, or —Se—,
R¹ to R⁶ each independently represent a hydrogen atom, a halogen atom, an alkyl group which may have a substituent, an alkoxy group which may have a substituent, an alkylthio group which may have a substituent, a silyl group which may have a substituent, an alkenyl group which may have a substituent, an alkynyl group which may have a substituent, an aryl group which may have a substituent, or a heteroaryl group which may have a substituent, and
Ar¹ and Ar² each independently represent an aromatic ring group which may have a substituent.

14. The photoelectric conversion element according to claim 13, wherein X², Y^{b1}, and Y^{b2} represent —S—.

15. The photoelectric conversion element according to claim 2, wherein Ar¹ and Ar² each independently represent a polycyclic aromatic hydrocarbon ring group which may have a substituent or a group represented by Formula (R),
—Ar^{X}—Ar^{Y} (R)
in formula (R), Ar^{X} represents a monocyclic aromatic ring group which may have a substituent in addition to Ar^{Y}, and
Ar^{Y} represents an aromatic ring group which may have a substituent.

16. The photoelectric conversion element according to claim 2, wherein the compound represented by Formula (1) has a molecular weight of 400 to 900.

17. The photoelectric conversion element according to claim 2, wherein the photoelectric conversion film has a bulk hetero structure formed in a state where the compound represented by Formula (1) and the n-type semiconductor material are mixed to each other.

18. The photoelectric conversion element according to claim 2, further comprising one or more interlayers between the conductive film and the transparent conductive film, in addition to the photoelectric conversion film.

19. The photoelectric conversion element according to claim 2, wherein the n-type semiconductor material includes fullerenes selected from the group consisting of a fullerene and a derivative thereof.

20. An imaging element comprising the photoelectric conversion element according to claim 2.
